(19) United States
(12) Patent Application Publication
(10) Pub. No.: US 2013/0190856 A1
(43) Pub. Date: Jul. 25, 2013

(54) METHODS AND APPARATUS FOR STENTING COMPRISING ENHANCED EMBOLIC PROTECTION COUPLED WITH IMPROVED PROTECTIONS AGAINST RESTENOSIS AND THROMBUS FORMATION

(71) Applicants: Randolph von Oepen, Aptos, CA (US); Gerd Seibold, Ammerbuch (DE); Silvio R. Schaffner, Berlingen (CH); Marc G Gianotti, Wiesendangen (CH); Joost J. Fierens, Dworp (BE); Erhard Huesler, Burgdorf (CH); Arik Zucker, Zurich (CH); Eric Marcoux, Erquelinnes (BE); Philippe Nicaise, Uccle (BE); Sebastien Dubois, Bois d'Haine (BE)

(72) Inventors: Randolph von Oepen, Aptos, CA (US); Gerd Seibold, Ammerbuch (DE); Silvio R. Schaffner, Berlingen (CH); Marc G Gianotti, Wiesendangen (CH); Joost J. Fierens, Dworp (BE); Erhard Huesler, Burgdorf (CH); Arik Zucker, Zurich (CH); Eric Marcoux, Erquelinnes (BE); Philippe Nicaise, Uccle (BE); Sebastien Dubois, Bois d'Haine (BE)

(73) Assignee: Abbott Laboratories Vascular Enterprises Limited, Dublin (IE)

(21) Appl. No.: 13/801,469

(22) Filed: Mar. 13, 2013

Related U.S. Application Data
(63) Continuation-in-part of application No. 12/895,032, filed on Sep. 30, 2010.
Application No. 12/895,032 is a continuation of application No. 11/313,110, filed on Dec. 19, 2005, now Pat. No. 7,815,763.
Application No. 11/313,110 is a continuation-in-part of application No. 10/859,636, filed on Jun. 3, 2004, now Pat. No. 7,927,364.
Application No. 10/859,636 is a continuation of application No. 09/967,789, filed on Sep. 28, 2001, now Pat. No. 6,755,856.
Application No. 09/967,789 is a continuation-in-part of application No. 09/742,144, filed on Dec. 19, 2000, now Pat. No. 6,682,554.
Application No. 09/742,144 is a continuation-in-part of application No. 09/582,318, filed on Jun. 23, 2000, now Pat. No. 6,602,285.
Application No. 09/582,318 is a continuation-in-part of application No. 13/089,039, filed on Apr. 18, 2011.
Application No. 13/089,039 is a continuation of application No. 11/731,820, filed on Mar. 29, 2007, now Pat. No. 7,927,365.
Application No. 11/731,820 is a continuation of application No. 10/859,636, filed on Jun. 3, 2004, now Pat. No. 7,927,364.
Application No. 10/859,636 is a continuation of application No. 09/967,789, filed on Sep. 28, 2001, now Pat. No. 6,755,856.
Application No. 09/967,789 is a continuation-in-part of application No. 09/742,144, filed on Dec. 19, 2000, now Pat. No. 6,682,554.
Application No. 09/742,144 is a continuation-in-part of application No. 09/582,318, filed on Jun. 23, 2000, now Pat. No. 6,602,285.
(60) Provisional application No. 60/637,495, filed on Dec. 20, 2004.

(30) Foreign Application Priority Data
Sep. 5, 1998 (DE) 19840645.2

(51) Int. Cl. A61F 2/06

(52) U.S. Cl. CPC A61F 2/06
USPC 623/1.15; 623/1.35; 623/1.42; 156/77; 156/350

(57) ABSTRACT

Apparatus and methods for stenting are provided comprising a stent attached to a porous biocompatible material that is permeable to endothelial cell ingrowth, but impermeable to release of emboli of predetermined size. Apparatus and methods are also provided for use at a vessel branching. The present invention further involves porous polymer membranes, suitable for use in medical implants, having controlled pore sizes, pore densities and mechanical properties. Methods of manufacturing such porous membranes are described in which a continuous fiber of polymer is extruded through a reciprocating extrusion head and deposited onto a substrate in a predetermined pattern. When cured, the polymeric material forms a stable, porous membrane suitable for a variety of applications, including reducing emboli release during and after stent delivery, and providing a source for release of bioactive substances to a vessel or organ and surrounding tissue.

### CROSS-REFERENCE TO RELATED APPLICATIONS

The present application claims priority as a continuation-in-part of U.S. patent application Ser. No. 12/895,032, filed Sep. 30, 2010, which is a continuation of U.S. patent application Ser. No. 11/313,110, filed Dec. 19, 2005, now U.S. Pat. No. 7,815,763, which is a continuation-in-part of U.S. patent application Ser. No. 10/859,636, filed Jun. 3, 2004, now U.S. Pat. No. 7,927,364. U.S. patent application Ser. No. 11/313,110 also claims the benefit of priority from U.S. Provisional Patent Application Ser. No. 60/637,495, filed Dec. 20, 2004. U.S. patent application Ser. No. 10/859,636 is a continuation of U.S. patent application Ser. No. 09/967,789, filed Sep. 28, 2001, now U.S. Pat. No. 6,755,856, which is a continuation-in-part of U.S. patent application Ser. No. 09/742,144, filed Dec. 19, 2000, now U.S. Pat. No. 6,682,554, which is a continuation-in-part of U.S. patent application Ser. No. 09/582,318, filed Jun. 23, 2000, now U.S. Pat. No. 6,602,285, which claims the benefit of and priority to International Application No. PCT/EP99/06456, filed Sep. 2, 1999, which claims the benefit of and priority to German Patent Application No. 19840645.2, filed Sep. 5, 1998. The above listed applications are incorporated herein by reference in their entireties.

The present application additionally claims priority as a continuation-in-part of U.S. patent application Ser. No. 13/089,039, filed Apr. 18, 2011, which is a continuation of U.S. patent application Ser. No. 11/731,820, filed Mar. 29, 2007, now U.S. Pat. No. 7,927,365, which is a continuation of U.S. patent application Ser. No. 10/859,636, filed Jun. 3, 2004, now U.S. Pat. No. 7,927,364, which is a continuation of U.S. patent application Ser. No. 09/967,789, filed Sep. 28, 2001, now U.S. Pat. No. 6,755,856, which is a continuation-in-part of U.S. patent application Ser. No. 09/742,144, filed Dec. 19, 2000, now U.S. Pat. No. 6,682,554, which is a continuation-in-part of U.S. patent application Ser. No. 09/582,318, filed Jun. 23, 2000, now U.S. Pat. No. 6,602,285, which claims the benefit of and priority to International Application No. PCT/EP99/06456, filed Sep. 2, 1999, which claims the benefit of and priority to German Patent Application No. 19840645.2, filed Sep. 5, 1998. The above listed applications are incorporated herein by reference in their entireties.

### FIELD OF THE INVENTION

The present invention relates to stents, and more particularly, to stent grafts having an expandable web structure configured to provide enhanced embolic protection and reduce restenosis and thrombus formation. The present invention additionally relates to porous membranes suitable for covering medical implants such as stents for intravascular delivery, implants covered with such membranes and methods for making the porous membranes.

### BACKGROUND OF THE INVENTION

Stents are commonly indicated for a variety of intravascular and non-vascular applications, including restoration and/or maintenance of patency within a patient's vessel. Stents are also used to reduce restenosis of a blood vessel post-dilation, thereby ensuring adequate blood flow through the vessel. Previously known stents are formed of a cell or mesh structure, having apertures through which endothelial cells migrate rapidly. These endothelial cells form a smooth coating over the stent that limits interaction between the stent and blood flowing through the vessel, thereby minimizing restenosis and thrombus formation.

In many applications, in addition to maintenance of vessel patency and limitation of restenosis, protection against release of embolic material from the walls of the vessel is desired. Emboli released into the bloodstream flow downstream, where they may occlude flow and cause death, stroke, or other permanent injury to the patient. The apertures between adjoining cells of previously known stents may provide an avenue for such embolic release, depending upon the application.

In addition to embolic protection, a smooth surface, i.e. a substantially continuous surface lacking apertures, may be desired to permit unencumbered recrossability with guide wires, balloon catheters, etc., into the lumen of the stent, for example, to compress stenosis or restenosis and open the lumen, to resize the stent to accommodate vascular geometry changes, etc. Further, equalization of forces applied by or to the stent may be desired to reduce a risk of the stent causing vessel dissection. Due to the apertures, previously known stents may provide only limited embolic protection, recrossability, and force distribution in some applications.

A covered stent, or a stent graft, comprises a stent that is at least partially externally-covered, internally-lined, or sintered with a biocompatible material that is impermeable to stenotic emboli. Common covering materials include biocompatible polymers, such as Polyethylene Terephthalate (PETP or “Dacron”) or expanded Polytetrafluoroethylene (ePTFE or “Teflon”). Stent grafts may be either balloon-expandable or self-expanding. Balloon-expandable systems may be expanded to an optimal diameter in-vivo that corresponds to the internal profile of the vessel. Upon compression, self-expanding embodiments characteristically return in a resilient fashion to their unstressed deployed configurations and are thus preferred for use in tortuous anatomy and in vessels that undergo temporary deformation.

A stent graft provides embolic protection by sealing emboli against a vessel wall and excluding the emboli from blood flow through the vessel. Additionally, since the biocompatible material of a stent graft closely tracks the profile of the stent, forces applied by and to an impinging vessel wall are distributed over a larger surface area of the stent, i.e. the force is not just applied at discrete points by “struts” located between apertures of the stent. Rather, the biocompatible material also carries the load and distributes it over the surface of the stent. Furthermore, stent grafts provide a smooth surface that allows improved or unencumbered recrossability into the lumen of the graft, especially when the biocompatible material lines the interior of, or is sintered into, the stent.

While the biocompatible materials used in stent grafts are impermeable to, and provide protection against, embolic release, they typically do not allow rapid endothelialization, as they also are impermeable or substantially impermeable to ingrowth of endothelial cells (i.e. have pores smaller than about 30 μm) that form the protective intime layer of blood vessels. These cells must migrate from the open ends of a stent graft into the interior of the stent. Migration occurs through blood flow and through the scaffold provided by the graft. Such migration is slow and may take a period of months, as opposed to the period of days to weeks required by bare (i.e. non-covered) stents.

In the interim, thrombus may form within the lumen of the graft, with potentially dire consequences. As a further drawback, migration of the endothelium through the open ends of a graft may leave the endothelial coating incomplete, i.e. it does not span a mid-portion of the graft. In addition, the endothelial layer is often thicker and more irregular than the endothelialization observed with bare stents, enhancing the risk of restenosis and thrombus formation.

Porous covered stents also are known. For example, U.S. Pat. No. 5,769,884 to Solovay describes a covered stent having porous regions near the end of the stent, wherein the pores are sized to allow tissue ingrowth and endothelialization. The middle region of the stent is described as being much less porous or non-porous, to encapsulate damaged or diseased tissue and inhibit tissue ingrowth.

The Solovay device is believed to have several drawbacks. First, the end regions of the stent are described as having a preferred pore diameter as large as 120 μm. However, pore diameters greater than about 100 μm may provide inadequate embolic protection; thus, if the end regions compress a stenosis, hazardous embolization may result. Second, since the middle region of the stent is adapted to inhibit tissue ingrowth, endothelial cells must migrate into the middle region of the stent from the end regions and from blood flow. As discussed previously, such migration is slow and provides an inferior endothelial layer.

An additional drawback to previously known devices is that many are not configured for use at a vessel bifurcation. A bare stent placed across a vessel side branch is expected to disrupt flow into the side branch and create turbulence that may lead to thrombus formation. Conversely, placement of a non-porous covered stent/stent graft across the bifurcation is expected to permanently exclude the side branch from blood flow, as such grafts are substantially impermeable to blood.

Covered stents for implantation into a body vessel, duct or lumen generally include a stent and a cover attached to the stent. A porous structure of the cover, depending on the porosity, may enhance tissue ingrowth after the covered stent has been implanted. A porous structure affixed to an implantable device also may serve as a reservoir for bioactive components and/or reduce embolization by trapping thrombus against a vessel wall.

Porous membranes for use in medical devices are known in the art. For example, U.S. Pat. No. 4,759,757 to Pinchuk describes the formation of a porous membrane by leaching water soluble inorganic salts incorporated into the membrane to create pores where the salt crystals were initially located. U.S. Pat. No. 6,540,776 to Sanders Millare et al. describes a perforated membrane in which a pattern of interstices is created by removing material, for example, by laser cutting. The foregoing manufacturing methods require at least two process steps to form a porous membrane.

One step processes for forming porous membranes also are known in the art, for example, using spinning techniques. U.S. Patent Application Publication No. 20040051201 to Greenhalgh et al. describes an electrospinning process in which a membrane is formed from a plurality of randomly-oriented, intertangled, non-woven fibrils.

Spinning techniques that produce less random, but non-uniform membranes, also are known. For example, U.S. Pat. No. 4,475,972 to Wong describes a porous polymeric material made by a process in which polymeric fibers are wound on a mandrel in multiple overlying layers. The fibers contain unevaporated solvent when deposited in contact with one another, so that upon evaporation of the solvent the fibers bond together. The fibers laid in one traverse are wound on the mandrel parallel to each other and at an angle with respect to the axis of the mandrel. In the next traverse, the angle of winding is reverse to the previous angle, so that the fibers crisscross each other in multiple layers to form the porous structure.

U.S. Pat. No. 4,738,740 to Pinchuk et al. describes a spinning method similar to that of Wong and further comprising intermittently applying a electrostatic charge to ensure reattachment of broken fibers to the mandrel. U.S. Pat. No. 5,653,747 to Dereume describes a vascular graft with an expandable coating produced by the spinning technique of Wong and having pores that open when the tubular support member expands.

All of the foregoing spinning processes suffer from an inability to tightly control the pore size and pore pattern of the resulting membranes. More specifically, lateral deviation of the fibers using previously known spinning techniques has resulted in unsteady collocation of the fibers and the need to deposit multiple layers to ensure adequate coverage. Consequently, previously-known techniques produce either stiff membranes formed of multiple layers and unsatisfactory porosity, or porous, elastic membranes with insufficient strength.

In view of the drawbacks associated with previously known stents and stent grafts, it would be desirable to provide apparatus and methods for stenting that overcome the drawbacks of previously known devices.

It further would be desirable to provide methods and apparatus that reduce the risk of embolic release, while also reducing the risk of restenosis and thrombus formation.

It also would be desirable to provide apparatus and methods for stenting that allow improved recrossability into the lumen of the apparatus.

It would be desirable to provide apparatus and methods for stenting that distribute forces applied by or to the apparatus.

It still further would be desirable to provide apparatus and methods suitable for use in bifurcated vessels.

Additionally, it would be desirable to provide membranes having controlled porosity, pore pattern and pore distribution.

It further would be desirable to provide a one step manufacturing process to produce membranes having controlled porosity, pore pattern and pore distribution.

It still further would be desirable to provide a one step manufacturing process to produce membranes having controlled porosity and/or pore pattern wherein the membrane includes a bioactive substance that may be eluted from the membrane after implantation.

It also would be desirable to provide manufacturing processes to produce membranes having the desired porosity, pattern and distribution characteristics for use in medical implants.

### SUMMARY OF THE INVENTION

In view of the foregoing, it is an object of the present invention to provide a stent that experiences reduced foreshortening during deployment.

It is another object to provide a stent that is flexible, even in the contracted delivery configuration.

It is also an object to provide a stent having radial stiffness in the expanded deployed configuration sufficient to maintain vessel patency in a stenosed vessel.

In view of the foregoing, it is an object of the present invention to provide apparatus and methods for stenting that overcome the drawbacks of previously known apparatus and methods.

It is an object to reduce the risk of embolic release during and after stenting, and also reduce the risk of restenosis and thrombus formation.

It is yet another object of the present invention to provide apparatus and methods that allow unencumbered recrossability into the lumen of the apparatus.

It is an object to provide apparatus and methods for stenting that distribute forces applied by or to the apparatus.

It is an object to provide apparatus and methods suitable for use in a bifurcated vessel.

These and other objects of the present invention are accomplished by providing apparatus comprising a stent, for example, a balloon-expandable, a self-expanding, a bistable cell, or a metal mesh stent. A biocompatible material at least partially is sintered between the apertures of the stent, or covers the interior or exterior surface (or both) of the stent. Unlike previously known stent grafts, embodiments of the present invention are both permeable ingrowth and impermeable to release of critical-sized emboli along their entire lengths. Thus, the present invention provides the embolic protection, force distribution, and improved recrossability characteristic of non-porous stent grafts, while further providing the protection against restenosis and thrombus formation characteristic of bare stents.

In one preferred embodiment, the biocompatible material of the present invention comprises, for example, a porous woven, knitted, or braided material having pore sizes determined as a function of the tightness of the weave, knit, or braid. Pore size is selected to allow endothelial cell ingrowth, while preventing release of emboli larger than a predetermined size. In an alternative embodiment, the biocompatible material comprises pores that are chemically, physically, mechanically, laser-cut, or otherwise created through the material with a specified diameter, spacing, etc. The pores may be provided with uniform or non-uniform density, size, and/or shape. The pores preferably have a minimum width large enough to promote endothelial cell ingrowth, and a maximum width small enough to reduce the risk of embolic release.

Apparatus also is provided for use in a bifurcated or branched vessel. Since the porous biocompatible material of the present invention is permeable to blood flow, it is expected that, when implanted, flow into a side branch will continue uninterrupted. The small diameter of the pores, relative to the diameter of the stent apertures, will provide a grating that is expected to minimize turbulence and allow thrombus-free blood flow into the side branch. Optionally, the porosity, i.e. the diameter, density, shape, and/or arrangement, of the pores may be altered in the region of the side branch to ensure adequate flow.

Alternatively, the stent and biocompatible material may comprise a radial opening. When stenting at a vessel bifurcation or branching, the radial opening may be positioned in line with the side branch to maintain patency of the branch. Alternatively, a plurality of radial openings may be provided along the length of the implant to facilitate continuous blood flow through a plurality of side branches.

Stents for use with apparatus of the present invention preferably comprise a tubular body with a wall having a web structure configured to expand from a contracted delivery configuration to an expanded deployed configuration. The web structure comprises a plurality of neighboring web patterns having adjoining webs. Each web has three sections: a central section arranged substantially parallel to the longitudinal axis in the contracted delivery configuration, and two lateral sections coupled to the ends of the central section. The angles between the lateral sections and the central section increase during expansion, thereby reducing or substantially eliminating length decrease of the stent due to expansion, while increasing a radial stiffness of the stent.

Preferably, each of the three sections of each web is substantially straight, the lateral sections preferably define obtuse angles with the central section, and the three sections are arranged relative to one another to form a concave or convex structure. When contracted to its delivery configuration, the webs resemble stacked or nested bowls or plates. This configuration provides a compact delivery profile, as the webs are packed against one another to form web patterns resembling rows of the stacked plates.

Neighboring web patterns are preferably connected to one another by connection elements preferably formed as straight sections. In a preferred embodiment, the connection elements extend between adjacent web patterns from the points of interconnection between neighboring webs within a given web pattern.

The orientation of connection elements between a pair of neighboring web patterns preferably is the same for all connection elements disposed between the pair. However, the orientation of connection elements alternates between neighboring pairs of neighboring web patterns. Thus, a stent illustratively flattened and viewed as a plane provides an alternating orientation of connection elements between the neighboring pairs: first upwards, then downwards, then upwards, etc.

As will be apparent to one of skill in the art, positioning, distribution density, and thickness of connection elements and adjoining webs may be varied to provide stents exhibiting characteristics tailored to specific applications. Applications may include, for example, use in the coronary or peripheral (e.g. renal) arteries. Positioning, density, and thickness may even vary along the length of an individual stent in order to vary flexibility and radial stiffness characteristics along the length of the stent.

Stents for use with apparatus of the present invention preferably are flexible in the delivery configuration. Such flexibility beneficially increases a clinician's ability to guide the stent to a target site within a patient's vessel. Furthermore, stents of the present invention preferably exhibit high radial stiffness in the deployed configuration. Implanted stents therefore are capable of withstanding compressive forces applied by a vessel wall and maintaining vessel patency. The web structure described hereinabove provides the desired combination of flexibility in the delivery configuration and radial stiffness in the deployed configuration. The combination further may be achieved, for example, by providing a stent having increased wall thickness in a first portion of the stent and decreased wall thickness with fewer connection elements in an adjacent portion or portions of the stent.

Embodiments of the present invention may comprise a coating or attached active groups configured for localized delivery of radiation, gene therapy, medicaments, thrombin inhibitors, or other therapeutic agents. Furthermore, embodiments may comprise one or more radiopaque features to facilitate proper positioning within a vessel.

Methods of using the apparatus of the present invention also are provided.

It is also an object of the present invention to provide membranes for use in medical implants having controlled porosity, pore pattern and pore distribution.

It is another object of this invention to provide a one-step manufacturing process to produce membranes having controlled porosity, pore pattern and pore distribution.

It is a further object of the present invention to provide a one-step manufacturing process to produce membranes having controlled porosity and/or pore pattern wherein the membrane includes a bioactive substance that may be eluted from the membrane after implantation.

It is also an object of this invention to provide manufacturing processes to produce membranes having the desired porosity, pattern and distribution characteristics for use in medical implants.

These and other objects of the present invention are accomplished by providing a membrane comprising a plurality of fibers that are deposited onto a substrate with a predetermined and reproducible pattern. The substrate may be either a mandrel or a surface of an implantable device, such as a stent. In a preferred embodiment, the fibers comprise a polymer that is sufficiently elastic and robust that the membrane follows the movements of the stent from loading onto a stent delivery system to deployment and implantation, without adversely affecting the performance of the membrane of the stent.

In a preferred embodiment, the membrane is formed using a computer-controller substrate that moves in a precisely controlled and reproducible manner. The polymer used to form the fibers, e.g., a polyurethane or a copolymer thereof, is dissolved in a solvent and extruded through one or more extrusion heads onto a moving substrate. By moving the extrusion head back and forth with a specific velocity along the axis of the substrate, specific filament angles or patterns may be deposited. In accordance with one aspect of the present invention, the number of passes, substrate shape and motion and extrusion head speed and material flow are controlled to provide a predetermined fiber diameter that is deposited to produce desired membrane properties, such as pore size and density.

The membrane may either be fixed on the exterior surface of an implantable device, such as a stent, on the interior surface or both. Where an exterior covering is desired, the membrane may be directly deposited on the implantable device. Alternatively, the covering may be deposited on a mandrel to form a separate component, and then affixed to the implantable device in a later manufacturing step.

In accordance with another aspect of the present invention, the membrane may comprise composite fibers having a viscous sheath co-extruded around a solid core component, or alternatively may comprise co-extruded viscous components. In this manner, a membrane may be created wherein the individual fibers are loaded with a desired bioactive agent, such as a drug, that elutes from the matrix of the membrane without resulting in substantial degradation of the mechanical properties of the membrane.

Methods of manufacturing covered implantable medical devices including the porous membranes of the present invention also are provided.

### BRIEF DESCRIPTION OF THE DRAWINGS

Further features of the invention, its nature and various advantages, will be more apparent from the following detailed description of the preferred embodiments, taken in conjunction with the accompanying drawings, in which like reference numerals apply to like parts throughout, and in which:

FIG. 1 is a schematic isometric view illustrating the basic structure of a preferred stent for use with apparatus of the present invention;

FIG. 2 is a schematic view illustrating a web structure of a wall of the stent of FIG. 1 in a contracted delivery configuration;

FIG. 3 is a schematic view illustrating the web structure of the stent of FIG. 1 in an expanded deployed configuration;

FIG. 4 is an enlarged schematic view of the web structure in the delivery configuration;

FIG. 5 is a schematic view of an alternative web structure of the stent of FIG. 1 having transition sections and shown in an as-manufactured configuration;

FIGS. 6A and 6B are, respectively, a schematic view and detailed view of an alternative embodiment of the web structure of FIG. 5;

FIGS. 7A-7D are, respectively, schematic and detailed views of another alternative embodiment of the web structure of the stent of the present invention, and a cross-sectional view of the stent;

FIGS. 8A and 8B are views further alternative embodiments of the stent of the present application having different interconnection patterns;

FIGS. 9A and 9B are, respectively, a schematic and detailed view of yet another alternative embodiment of the web structure of FIG. 5;

FIGS. 10A-10D illustrate a method of deploying a balloon expandable embodiment of a stent constructed in accordance with the present invention;

FIGS. 11A-11C are side-sectional views of a prior art bare stent in an expanded deployed configuration within a patient's vasculature, illustrating limitations of bare stents with regard to embolic protection, recrossability, and force distribution, respectively;

FIG. 12 is a side-sectional view of a prior art, non-porous stent graft in an expanded deployed configuration within a patient's vasculature, illustrating the potential for thrombus formation and restenosis due to inefficient endothelial cell migration;

FIGS. 13A and 13B are side-sectional views of a first embodiment of apparatus of the present invention, shown, respectively, in a collapsed delivery configuration and in a deployed configuration;

FIGS. 14A-14D are side-sectional views of the apparatus of FIG. 13 within a patient's vasculature, illustrating a method of using the apparatus in accordance with the present invention;

FIGS. 15A-15C are side-sectional views of the apparatus of FIG. 13 within a patient's vasculature, illustrating capacity for reintroduction into the lumen of the apparatus and a method for establishing or restoring vessel patency after implantation of the apparatus;

FIG. 16 is a side-sectional view of the apparatus of FIG. 13 within a patient's vasculature illustrating force distribution upon interaction with an impinging vessel;

FIG. 17 is a side-sectional view of the apparatus of FIG. 13 in use at a vessel branching;

FIG. 18 is a side-sectional view of an alternative embodiment of apparatus of the present invention comprising a radial opening, in use at a vessel branching;

FIGS. 19A and 19B are cross-sectional views, illustrating stent/stent covering attachment schemes;

FIGS. 20A-20D are isometric schematic views illustrating various techniques for attaching a stent covering to a stent in a manner that provides the attachment scheme of FIG. 19B;

FIG. 21 is a schematic depiction of a membrane manufacturing system constructed in accordance with the principles of the present invention;

FIGS. 22A-22C are perspective views depicting exemplary patterns for depositing fibers onto a moving substrate in accordance with the present invention;

FIG. 23 is a perspective view illustrating a stent covered with the membrane of the present invention;

FIG. 24 is a schematic depiction of a membrane manufacturing process wherein the fibers comprise a core filament having a polymeric sheath; and

FIG. 25 is a schematic depiction of a membrane manufacturing process wherein the fibers comprise a coextrusion of two polymers.

### DETAILED DESCRIPTION OF THE INVENTION

The present invention relates to stent grafts having an expandable web structure, the stent grafts configured to provide enhanced embolic protection and improved protection against restenosis and thrombus formation. These attributes are attained by attaching to a stent a biocompatible material that is impermeable to emboli but permeable to ingrowth of endothelial cells. Attaching the material to the stent also distributes forces applied to or by the apparatus, and facilitates recrossing into the lumen of the apparatus post-implantation with guide wires, balloons, etc. Thus, unlike previously known bare stents, the present invention provides improved protection against embolic release, a smoother surface for recrossing, and better distribution of forces applied to or by the apparatus. Moreover, unlike previously known, non-porous stent grafts, the present invention provides enhanced protection against thrombus formation and restenosis via rapid endothelialization.

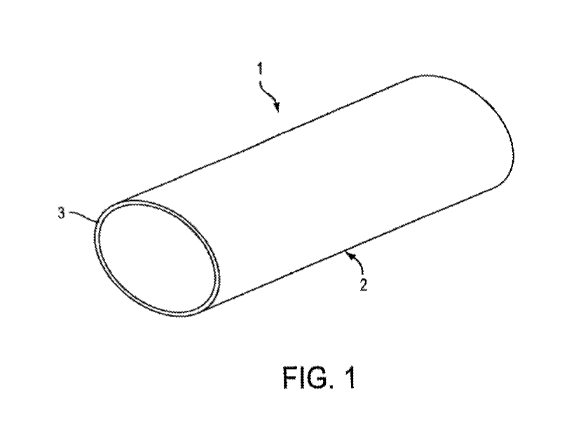

Prior to detailed presentation of embodiments of the present invention, preferred stent designs for use with such embodiments are provided in FIGS. 1-5. Stent 1 comprises tubular flexible body 2 having wall 3. Wall 3 comprises a web structure described herein below with respect to FIGS. 2-5.

Stent 1 and its web structure are expandable from a contracted delivery configuration to an expanded deployed configuration. Depending on the material of fabrication, stent 1 may be either self-expanding or expandable using a balloon catheter. If self-expanding, the web structure is preferably fabricated from a superelastic material, such as a nickel-titanium alloy. Regardless of the expansion mechanism used, the beneficial aspects of the present invention are maintained: reduced shortening upon expansion, high radial stiffness, and a high degree of flexibility. Furthermore, stent 1 preferably is fabricated from biocompatible and/or biodegradable materials. It also may be radiopaque to facilitate delivery, and it may comprise an external coating C that, for example, retards thrombus formation or restenosis within a vessel. The coating alternatively may deliver therapeutic agents into the patient's blood stream.

With reference to FIGS. 2-4, a first embodiment of the web structure of stent 1 is described. In FIGS. 2-4, wall 3 of body 2 of stent 1 is shown flattened into a plane for illustrative purposes. FIG. 2 shows web structure 4 in a contracted delivery configuration, with line L indicating the longitudinal axis of the stent. Web structure 4 comprises neighboring web patterns 5 and 6 arranged in alternating, side-by-side fashion. Thus, the web patterns seen in FIG. 2 are arranged in the sequence 5, 6, 5, 6, 5, etc.

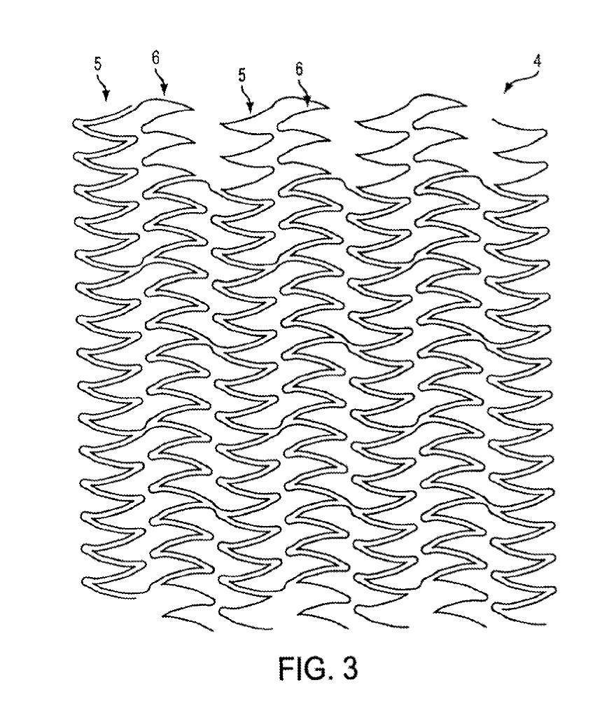

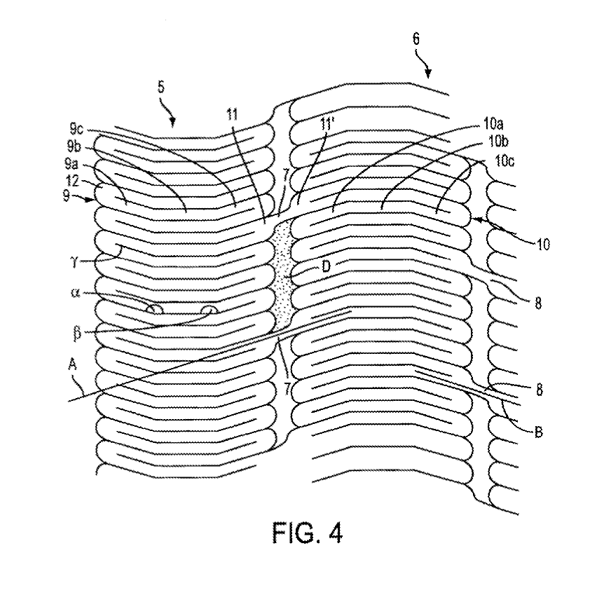

FIG. 2 illustrates that web patterns 5 comprise adjoining webs 9 (concave up in FIG. 2), while web patterns 6 comprise adjoining webs 10 (convex up in FIG. 2). Each of these webs has a concave or convex shape resulting in a stacked plate- or bowl-like appearance when the stent is contracted to its delivery configuration. Webs 9 of web patterns 5 are rotated 180 degrees with respect to webs 10 of web patterns 6, i.e., alternating concave and convex shapes. The structure of webs 9 and 10 is described in greater detail herein below with respect to FIG. 4.

Neighboring web patterns 5 and 6 are interconnected by connection elements 7 and 8. A plurality of connection elements 7 and 8 are provided longitudinally between each pair of web patterns 5 and 6. Multiple connection elements 7 and 8 are disposed in the circumferential direction between adjacent webs 5 and 6. The position, distribution density, and thickness of these pluralities of connection elements may be varied to suit specific applications in accordance with the present invention.

Connection elements 7 and 8 exhibit opposing orientation. However, all connection elements 7 preferably have the same orientation that, as seen in FIG. 2, extends from the left side, bottom, to the right side, top. Likewise, all connection elements 8 preferably have the same orientation that extends from the left side, top, to the right side, bottom. Connection elements 7 and 8 alternate between web patterns 5 and 6, as depicted in FIG. 2.

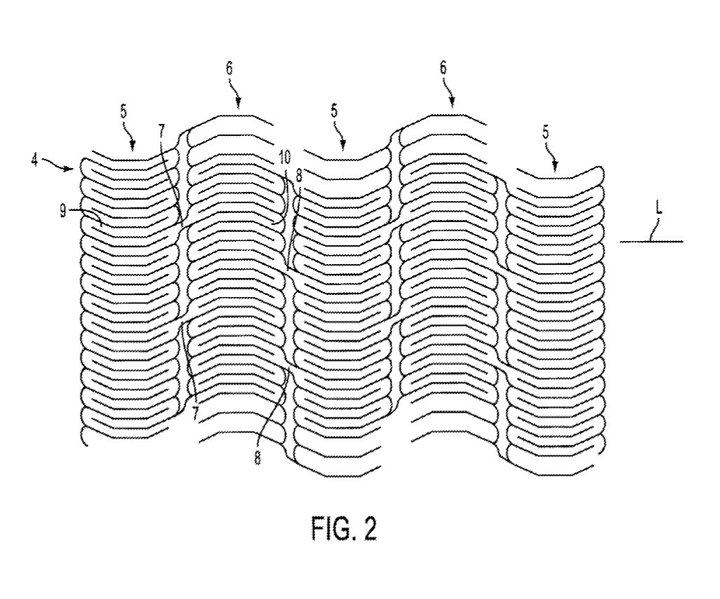

FIG. 3 illustrates the expanded deployed configuration of stent 1, again with reference to a portion of web structure 4, in an illustration where the wall 3 of the body 2 of the stent 1 is unwound into the plane of FIG. 3. When stent 1 is in the expanded deployed configuration, web structure 4 provides stent 1 with high radial stiffness. This stiffness enables stent 1 to remain in the expanded configuration while, for example, under radial stress. Stent 1 may experience application of radial stress when, for example, implanted into a hollow vessel in the area of a stenosis.

FIG. 4 is an enlarged view of web structure 4 detailing a portion of the web structure disposed in the contracted delivery configuration of FIG. 2. FIG. 4 illustrates that each of webs 9 of web pattern 5 comprises three sections 9a, 9b and 9c, and each of webs 10 of web pattern 6 comprises three sections 10a, 10b and 10c. Preferably, each individual section 9a, 9b, 9c, 10a, 10b and 10c, has a straight configuration.

Each web 9 has a central section 9b connected to lateral sections 9a and 9c, thus forming the previously mentioned bowl- or plate-like configuration. Sections 9a and 9b enclose obtuse angle .alpha. Likewise, central section 9b and lateral section 9c enclose obtuse angle .beta. Sections 10a-10c of each web 10 of each web pattern 6 are similarly configured, but are rotated 180 degrees with respect to corresponding webs 9. Where two sections 9a or 9c, or 10a or 10c adjoin one another, third angle gamma is formed (this angle is zero where the stent is in the fully contracted position, as shown in FIG. 4).

Preferably, central sections 9b and 10b are substantially aligned with the longitudinal axis L of the tubular stent, when the stent is in the contracted delivery configuration. The angles between the sections of each web increase in magnitude during expansion to the deployed configuration, except that angle .gamma., which is initially zero or acute, approaches a right angle after deployment of the stent. This increase provides high radial stiffness with reduced shortening of the stent length during deployment. As will of course be understood by one of ordinary skill in the art, the number of adjoining webs that span a circumference of the stent preferably is selected corresponding to the vessel diameter in which the stent is to be implanted.

FIG. 4 illustrates that, with stent 1 disposed in the contracted delivery configuration, webs 9 adjoin each other in an alternating fashion and are each arranged like plates stacked into one another, as are adjoining webs 10. FIG. 4 further illustrates that the configuration of the sections of each web applies to all of the webs, which jointly form web structure 4 of wall 3 of tubular body 2 of stent 1. Webs 9 are interconnected within each web pattern 5 via rounded connection sections 12, of which one connection section 12 is representatively labeled. Webs 10 of each neighboring web pattern 6 are similarly configured.

FIG. 4 also once again demonstrates the arrangement of connection elements 7 and 8. Connection elements 7, between a web pattern 5 and a neighboring web pattern 6, are disposed obliquely relative to the longitudinal axis L of the stent with an orientation A, which is the same for all connection elements 7. Orientation A is illustrated by a straight line that generally extends from the left side, bottom, to the right side, top of FIG. 4. Likewise, the orientation of all connection elements 8 is illustrated by line B that generally extends from the left side, top, to the right side, bottom of FIG. 4. Thus, an alternating A, B, A, B, etc., orientation is obtained over the entirety of web structure 4 for connection elements between neighboring web patterns.

Connection elements 7 and 8 are each configured as a straight section that passes into a connection section 11 of web pattern 5 and into a connection section 11′ of web pattern 6. This is illustratively shown in FIG. 4 with a connection element 7 extending between neighboring connection sections 11 and 11′, respectively. It should be understood that this represents a general case for all connection elements 7 and 8.

Since each web consists of three interconnected sections that form angles alpha and beta with respect to one another, which angles are preferably obtuse in the delivery configuration, expansion to the deployed configuration of FIG. 3 increases the magnitude of angles alpha and beta. This angular increase beneficially provides increased radial stiffness in the expanded configuration. Thus, stent 1 may be flexible in the contracted delivery configuration to facilitate delivery through tortuous anatomy, and also may exhibit sufficient radial stiffness in the expanded configuration to ensure vessel patency, even when deployed in an area of stenosis. The increase in angular magnitude also reduces and may even substantially eliminate length decrease of the stent due to expansion, thereby decreasing a likelihood that stent 1 will not completely span a target site within a patient's vessel post-deployment.

The stent of FIG. 4 is particularly well suited for use as a self-expanding stent when manufactured, for example, from a shape memory alloy such as nickel-titanium. In this case, web patterns 5 and 6 preferably are formed by laser-cutting a tubular member, wherein adjacent webs 9 and 10 are formed using slit-type cuts. Only the areas circumferentially located between connection members 7 and 8 (shaded area D in FIG. 4) require removal of areas of the tubular member. These areas also may be removed from the tubular member using laser-cutting techniques.

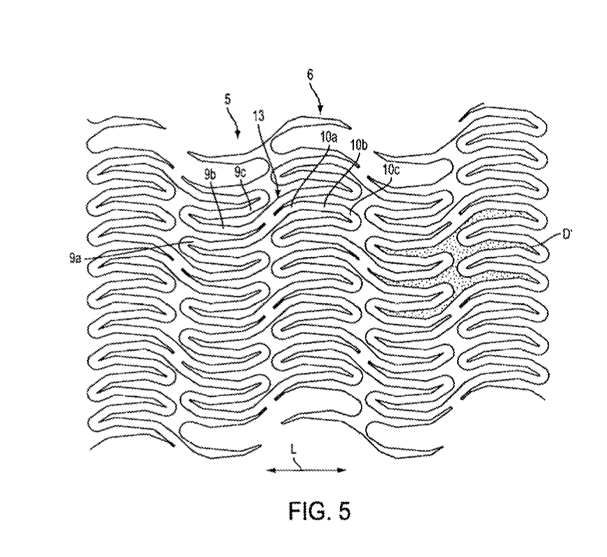

Referring now to FIG. 5, an alternative embodiment of the web structure of stent 1 is described. FIG. 5 shows the alternative web structure in an as-manufactured configuration. The basic pattern of the embodiment of FIG. 5 corresponds to that of the embodiment of FIGS. 2-4. Thus, this alternative embodiment also relates to a stent having a tubular flexible body with a wall having a web structure that is configured to expand from a contracted delivery configuration to the deployed configuration.

Likewise, the web structure again comprises a plurality of neighboring web patterns, of which two are illustratively labeled in FIG. 5 as web patterns 5 and 6. Web patterns 5 and 6 are again provided with adjoining webs 9 and 10, respectively. Each of webs 9 and 10 is subdivided into three sections, and reference is made to the discussion provided hereinabove, particularly with respect to FIG. 4. As will of course be understood by one of skill in the art, the stent of FIG. 5 will have a smaller diameter when contracted (or crimped) for delivery, and may have a larger diameter than illustrated in FIG. 5 when deployed (or expanded) in a vessel.

The embodiment of FIG. 5 differs from the previous embodiment by the absence of connection elements between web patterns. In FIG. 5, web patterns are interconnected to neighboring web patterns by transition sections 13, as shown by integral transition section 13 disposed between sections 9c and 10c. Symmetric, inverted web patterns are thereby obtained in the region of transition sections 13. To enhance stiffness, transition sections 13 preferably have a width greater than twice the width of webs 9 or 10.

As seen in FIG. 5, every third neighboring pair of webs 9 and 10 is joined by an integral transition section 13. As will be clear to those of skill in the art, the size and spacing of transition sections 13 may be altered in accordance with the principles of the present invention.

An advantage of the web structure of FIG. 5 is that it provides stent 1 with compact construction coupled with a high degree of flexibility in the delivery configuration and high load-bearing capabilities in the deployed configuration. Furthermore, FIG. 5 illustrates that, as with connection elements 7 and 8 of FIG. 4, transition sections 13 have an alternating orientation and are disposed obliquely relative to the longitudinal axis of the stent (shown by reference line L). FIG. 5 also illustrates that, especially in the deployed configuration, an H-like configuration of transition sections 13 with adjoining web sections is obtained.

The stent of FIG. 5 is well suited for use as a balloon-expandable stent, and may be manufactured from stainless steel alloys. Unlike the stent of FIG. 4, which is formed in the contracted delivery configuration, the stent of FIG. 5 preferably is formed in a partially deployed configuration by removing the shaded areas D′ between webs 9 and 10 using laser-cutting or chemical etching techniques. In this case, central sections 9b and 10b are substantially aligned with the longitudinal axis L of the stent when the stent is crimped onto the dilatation balloon of a delivery system.

As will be apparent to one of skill in the art, positioning, distribution density, and thickness of connection elements and adjoining webs may be varied to provide stents exhibiting characteristics tailored to specific applications. Applications may include, for example, use in the coronary or peripheral (e.g. renal) arteries. Positioning, density, and thickness may even vary along the length of an individual stent in order to vary flexibility and radial stiffness characteristics along the length of the stent.

Stents of the present invention preferably are flexible in the delivery configuration. Such flexibility beneficially increases a clinician's ability to guide the stent to a target site within a patient's vessel. Furthermore, stents of the present invention preferably exhibit high radial stiffness in the deployed configuration. Implanted stents therefore are capable of withstanding compressive forces applied by a vessel wall and maintain vessel patency. The web structure described hereinabove provides the desired combination of flexibility in the delivery configuration and radial stiffness in the deployed configuration. The combination further may be achieved, for example, by providing a stent having increased wall thickness in a first portion of the stent and decreased wall thickness with fewer connection elements in an adjacent portion or portions of the stent.

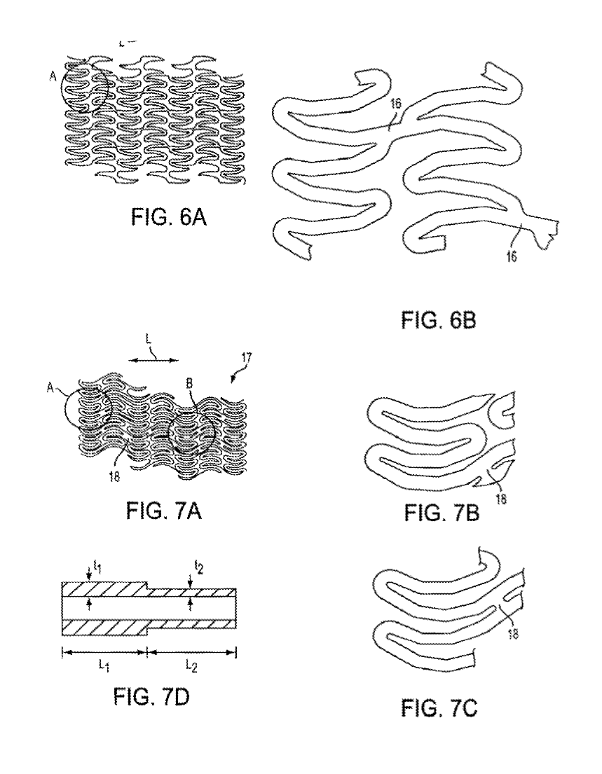

Referring now to FIGS. 6 and 7, alternative embodiments of the web structure of FIG. 5 are described. These web structures differ from the embodiment of FIG. 5 in the spacing of the transition sections. Web structure 15 of FIGS. 6A and 6B provides a spacing of transition sections 16 suited for use in the coronary arteries. FIG. 6A shows the overall arrangement, while FIG. 6B provides a detail view of region A of FIG. 6A. Other arrangements and spacings will be apparent to those of skill in the art and fall within the scope of the present invention.

Web structure 17 of FIGS. 7A-7D provides stent 1 with a variable wall thickness and a distribution density or spacing of transition sections 16 suited for use in the renal arteries. FIG. 7A shows the arrangement of web structure 17 along the length of stent 1, and demonstrates the spacing of transition sections 18. FIGS. 7C and 7D provide detail views of regions A and B, respectively, of FIG. 7A, showing how the spacing and shape of the webs that make up web structure 17 change as stent 1 changes along its length. In particular, as depicted (not to scale) in FIG. 7D, stent 1 has first thickness t.sub.1 for first length L.sub.1 and second thickness t.sub.2 for second length L.sub.2.

The variation in thickness, rigidity and number of struts of the web along the length of the stent of FIGS. 7A-7D facilitates use of the stent in the renal arteries. For example, the thicker region L.sub.1 includes more closely spaced and sturdier struts to provide a high degree of support in the ostial region, while the thinner region L.sub.2 includes fewer and thinner struts to provide greater flexibility to enter the renal arteries. For such intended applications, region L.sub.1 preferably has a length of about 6-8 mm and a nominal thickness t.sub.1 of 0.21 mm, and region L.sub.2 has a length of about 5 mm and a nominal thickness t.sub.2 of about 0.15 mm.

As depicted in FIGS. 7A-7D, the reduction in wall thickness may occur as a step along the exterior of the stent, such as may be obtained by grinding or chemical etching. One of ordinary skill in the art will appreciate, however, that the variation in thickness may occur gradually along the length of the stent, and that the reduction in wall thickness could be achieved by alternatively removing material from the interior surface of the stent, or both the exterior and interior surfaces of the stent.

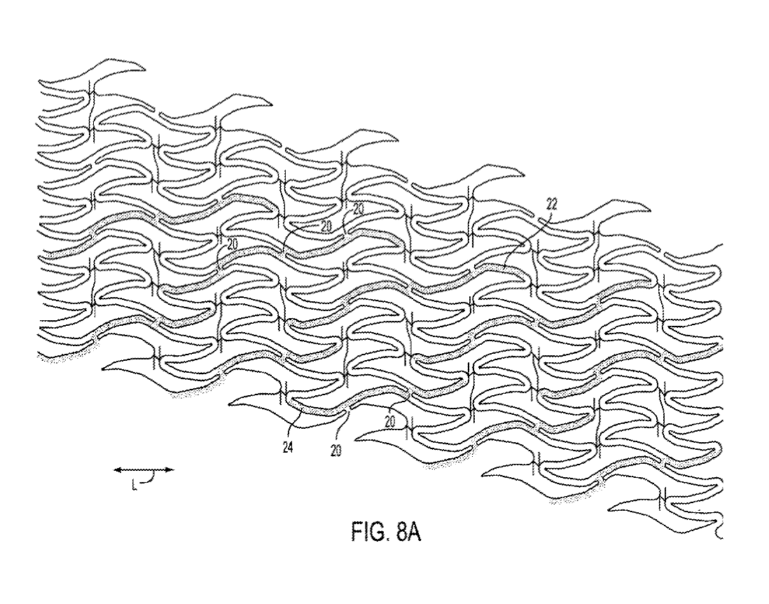

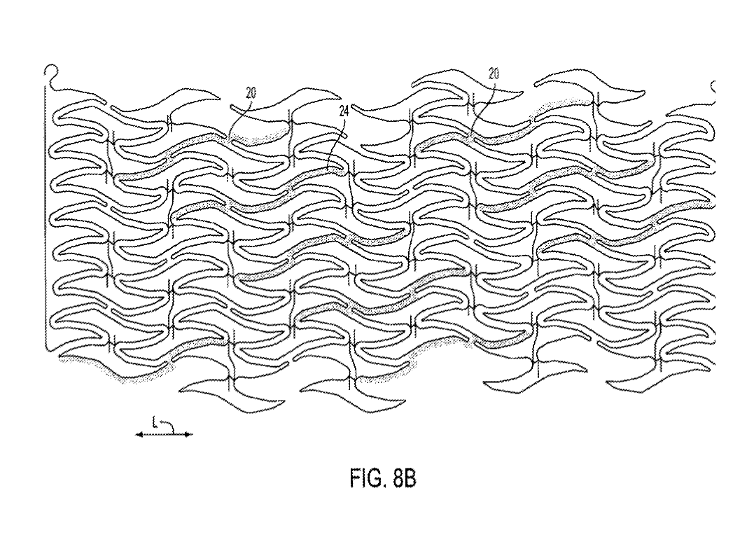

In FIGS. 8A and 8B, additional embodiments of web structures of the present invention, similar to FIG. 5, are described, in which line L indicates the direction of the longitudinal axis of the stent. In FIG. 5, every third neighboring pair of webs is joined by an integral transition section 13, and no set of struts 9a-9c or 10a-10c directly joins two transition sections 13. In the embodiment of FIG. 8A, however, integral transition sections 20 are arranged in a pattern so that the transition sections span either four or three adjacent webs. For example, the portion indicated as 22 in FIG. 8A includes three consecutively joined transition sections, spanning four webs. In the circumferential direction, portion 22 alternates with the portion indicated at 24, which includes two consecutive transition sections, spanning three webs.

By comparison, the web pattern depicted in FIG. 8B includes only portions 24 that repeat around the circumference of the stent, and span only three webs at a time. As will be apparent to one of ordinary skill, other arrangements of integral transition regions 13 may be employed, and may be selected on an empirical basis to provide any desired degree of flexibility and trackability in the contracted delivery configuration, and suitable radial strength in the deployed configuration.

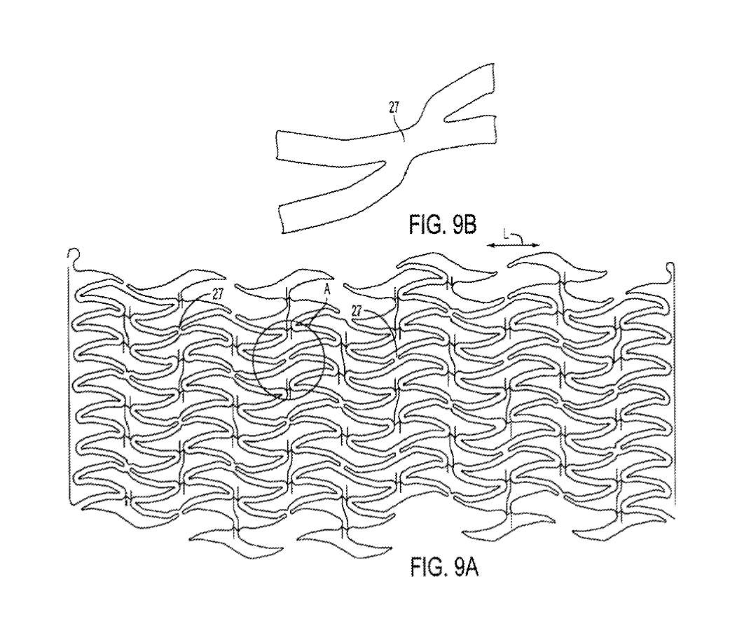

Referring now to FIGS. 9A and 9B, a further alternative embodiment of the stent of FIG. 8B is described, in which the transition sections are formed with reduced thickness. Web structure 26 comprises transition sections 27 disposed between neighboring web patterns. Sections 27 are thinner and comprise less material than transition sections 20 of the embodiment of FIG. 8B, thereby enhancing flexibility without significant reduction in radial stiffness.

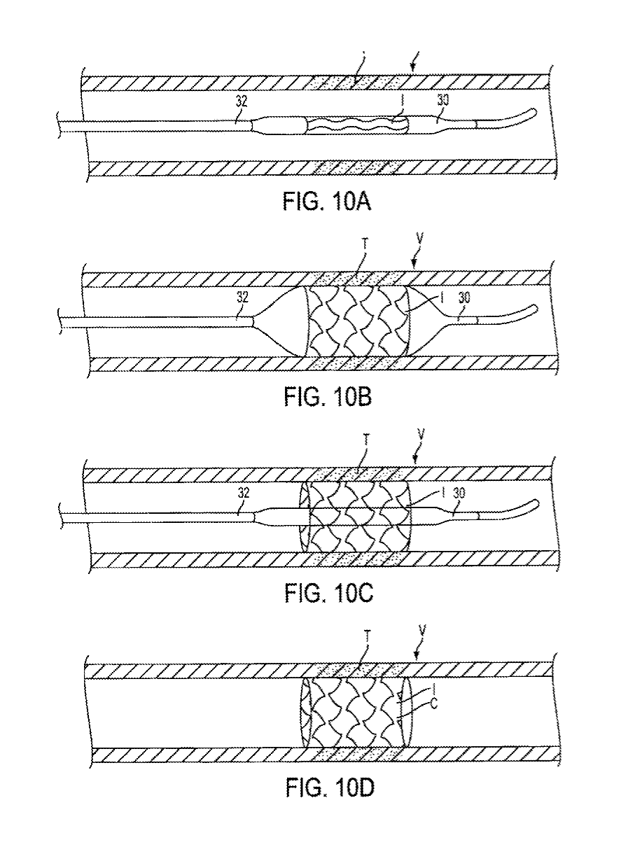

Referring now to FIGS. 10A-10D, a method of using a balloon expandable embodiment of stent 1 is provided. Stent 1 is disposed in a contracted delivery configuration over balloon 30 of balloon catheter 32. As seen in FIG. 10A, the distal end of catheter 32 is delivered to a target site T within a patient's vessel V using, for example, well-known percutaneous techniques. Stent 1 or portions of catheter 32 may be radiopaque to facilitate positioning within the vessel. Target site T may, for example, comprise a stenosed region of vessel V at which an angioplasty procedure has been conducted.

In FIG. 10B, balloon 30 is inflated to expand stent 1 to the deployed configuration in which it contacts the wall of vessel V at target site T. Notably, the web pattern of stent 1 described hereinabove minimizes a length decrease of stent 1 during expansion, thereby ensuring that stent 1 covers all of target site T. Balloon 30 is then deflated, as seen in FIG. 10C, and balloon catheter 32 is removed from vessel V, as seen in FIG. 10D.

Stent 1 is left in place within the vessel. Its web structure provides radial stiffness that maintains stent 1 in the expanded configuration and minimizes restenosis. Stent 1 may also comprise external coating C configured to retard restenosis or thrombosis formation around the stent. Coating C may alternatively deliver therapeutic agents into the patient's blood stream

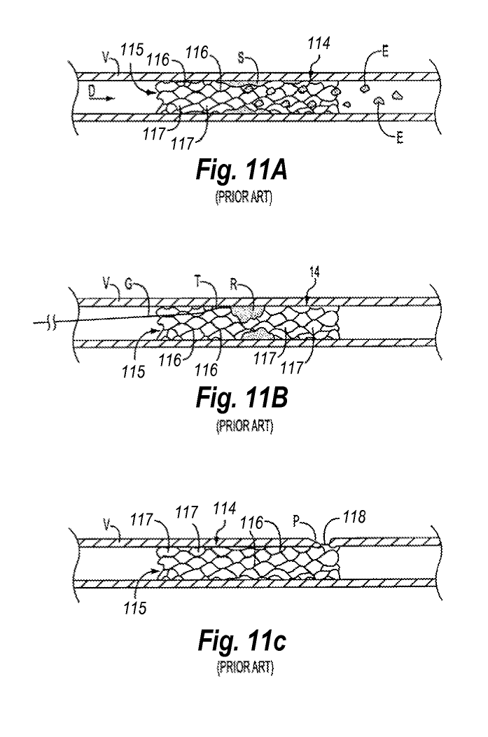

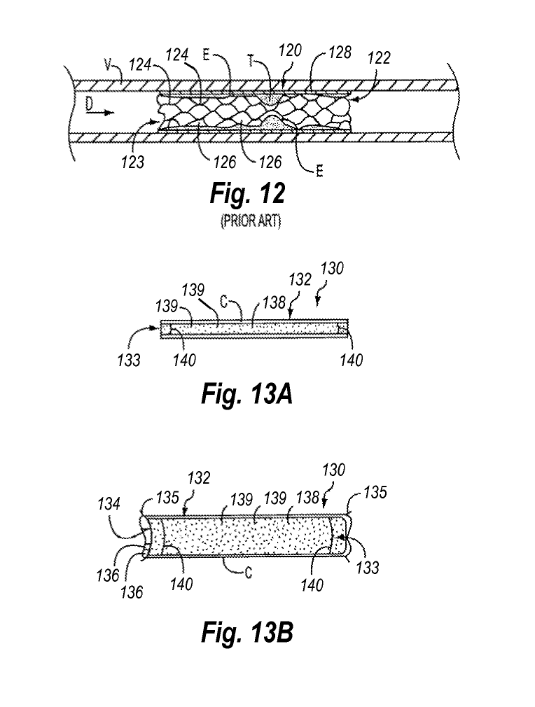

Referring now to FIGS. 11 and 12, limitations of previously known apparatus are described prior to detailed description of embodiments of the present invention. In FIGS. 11A-11C, limitations of a previously known bare stent are described. As seen in FIG. 11A, stent 114 has been implanted within a patient's vessel V at a treatment site exhibiting stenosis S, using, well-known techniques. Stent 114 has lumen 115 and comprises cell or mesh structure 116 having apertures 117. Stent 114 is shown expanded, e.g. either resiliently or via a balloon, to compress stenosis S against the wall of vessel V and restore patency within the vessel. During compression of stenosis S, particles have broken away from the stenosis to form emboli E. These emboli escape from the vessel wall through apertures 117 of stent 114. Blood flowing through vessel V in direction D carries the released emboli E downstream, where the emboli may occlude flow and cause death, stroke, or other permanent injury to the patient. Stent 114 therefore may provide inadequate embolic protection, depending upon the specific application.

In FIG. 11B, stent 114 has been implanted for an extended period of time in vessel V across a stenosed region. Restenosis R has formed within lumen 115 of stent 114, requiring further reintervention to restore patency to the vessel. Apertures 117 of stent 114 provide the stent with a non-uniform surface that complicates recrossing of the stent with guide wires, angioplasty balloons, etc., post-implantation.

In FIG. 11B, guide wire G has been advanced through the patient's vasculature into lumen 115 of stent 114 to provide a guide for advancement of an angioplasty balloon to compress restenosis R and reopen vessel V (not shown). Distal tip T of guide wire G has become entangled within structure 116 of stent 114 during recrossing, because the wire has inadvertently passed through an aperture 117. If guide wire G becomes caught on structure 116, emergency surgery may be necessary to remove the guide wire. Alternatively, a portion of guide wire G (or a portion of any other device inserted post-implantation through lumen 115 and entangled within stent 114) may break off from the guide wire and remain within the vessel, presenting a risk for thrombus formation or vessel dissection.

In addition to the problems associated with recrossing bare stent 114 upon restenosis, if stent 14 is self-expanding, the stent may provide inadequate radial force to compress a vessel stenosis at the time of implantation (not shown). Recrossing lumen 115 of stent 114 with a balloon catheter then may be necessary to compress the stenosis and fully open the lumen (not shown). As illustrated in FIG. 11B, such recrossing may be difficult or impossible.

In FIG. 11C, stent 14 has been implanted into vessel V that is subject to temporary deformation, for example, due to contact with neighboring muscles, due to joint motion, or due to external pressure applied to the vessel. The wall of vessel V impinges on a single strut 118 of structure 116 of stent 114. Since all force is concentrated at the point of impingement of vessel V and strut 118, strut 118 punctures vessel V at site P. Alternatively, temporary deformation of vessel V may kink stent 114 at strut 118, thus reducing lumen 115 and decreasing the utility of stent 114 (not shown). Clearly, either of these conditions may create a serious risk to the health of the patient. Similarly, stent 110 may dissect the vessel wall or may kink if implanted in tortuous anatomy (not shown). It would therefore be desirable to modify stent 114 to better distribute loads applied to the stent.

Referring now to FIG. 12, limitations of a previously known, non-porous covered stent, or stent graft, are described. Stent graft 120 comprises balloon-expandable or self-expanding stent 122 having lumen 123. Stent 122 comprises cell or mesh structure 124 having apertures 126. The stent is covered with biocompatible material 128, which commonly comprises a biocompatible polymer, such as PTFE, PETP, or a homologic material. Biocompatible material 128 is beneficially impermeable to stenotic emboli, but detrimentally impermeable to endothelial cell ingrowth.

In FIG. 12, graft 120 has been implanted for an extended period of time, for example, a period of months, within vessel V. Unlike stent 141 of FIG. 6, endothelial cells are not able to rapidly migrate through apertures 126 of stent 122 and surround graft 120 with a thin, uniform layer of endothelial cells that limit interaction between the graft and blood flowing through the vessel, thereby reducing restenosis and thrombus formation. Rather, since biocompatible material 128 is impermeable to ingrowth of the endothelial cells that form the protective intime layer of blood vessels, these cells must migrate from the open ends of graft 120 into the interior of lumen 123.

Migration occurs via blood flowing through vessel V in direction D and via the scaffold provided by the body of graft 120. However, this migration is slow and may take a period of months, as opposed to the period of days to weeks required for endothelialization of bare stents. Furthermore, as illustrated by endothelial layer E in FIG. 12, migration through the open ends of graft 120 may provide an incomplete endothelial layer, i.e. a layer that does not span a mid-portion of the graft. Layer E also may be thicker and more irregular than the endothelial layer obtained with bare stents. Gaps, irregularity, and thickening in layer E, as well as extended time required for formation of layer E, may yield thrombus T or restenosis within lumen 123 of graft 120, with potentially dire consequences. Stent graft 120 therefore may not provide adequate protection against restenosis and thrombus formation.

Referring now to FIGS. 13A and 13B, a first embodiment of apparatus of the present invention is described in greater detail. Apparatus 130 comprises stent 132 having lumen 133. Stent 132 may be, for example, self-expanding or balloon-expandable, or may be of bistable cell or metal mesh construction. Stent 132 comprises cell or mesh structure 134 with apertures 136. In a preferred embodiment, stent 132 comprises the design of stent 1, described hereinabove with respect to FIGS. 1-5. Stent 132 may further comprise an anchoring feature, such as hook or barb 135, to facilitate attachment to a vessel wall. The anchoring feature alternatively may comprise structure 134, which interacts with the vessel wall, for example, by pressing against the wall or by endothelial cell ingrowth into the structure, to anchor stent 132. Biocompatible material 138 having pores 139 is attached to at least a portion of stent 132.

Unlike material 128 of stent graft 120 (and unlike the material described hereinabove with respect to U.S. Pat. No. 5,769,884 to Solovay), material 138 of apparatus 130 is both permeable to endothelial cell ingrowth and impermeable to release of emboli of predetermined size, e.g. larger than about 100 μm, along its entire length. Thus, like stent graft 120 of FIG. 12, apparatus 130 provides enhanced embolic protection, improved force distribution, and improved recrossability; furthermore, like bare stent 114 of FIG. 11, apparatus 130 provides enhanced protection against restenosis and thrombus formation.

Biocompatible material 138 may comprise a biocompatible polymer, for example, a modified thermoplastic Polyurethane, Polyethylene Terephthalate, Polyethylene Tetraphthalate, expanded Polytetrafluoroethylene, Polypropylene, Polyester, Nylon, Polyethylene, Polyurethane, or combinations thereof. Alternatively, biocompatible material 138 may comprise a homologic material, such as an autologous or non-autologous vessel. Further still, material 138 may comprise a biodegradable material, for example, Polylactate or Polyglycolic Acid. In FIG. 13, material 138 illustratively lines the interior surface of stent 132, but it should be understood that material 138 alternatively may cover the stent's exterior surface, may be sintered within apertures 136 of stent 132, or may otherwise be attached to the stent.

Material 138 preferably comprises a woven, knitted, or braided material, wherein the size of pores 139 is determined as a function of the tightness of the weave, knit, or braid. The size of pores 139 then may be specified to allow endothelial cell ingrowth, while preventing release of emboli larger than a critical dangerous size, for example, larger than about 100 μm. In an alternative embodiment, the biocompatible material comprises pores 139 that are chemically, physically, mechanically, laser-cut, or otherwise created through material 138 with a specified diameter, spacing, etc.

Pores 139 may be provided with uniform or non-uniform density, size, and/or shape. The pores preferably have a minimum width no smaller than approximately 130 μm and a maximum width no larger than approximately 100 μm. Widths smaller than about 30 μm are expected to inhibit endothelial cell ingrowth, while widths larger than about 100 μm are expected to provide inadequate embolic protection, i.e. emboli of dangerous size may be released into the blood stream. Each of pores 139 is even more preferably provided with a substantially uniform, round shape having a diameter of approximately 80 μm. Pores 139 preferably are located along the entire length of material 138.

Stent 132 may be fabricated from a variety of materials. If self-expanding, the stent preferably comprises a superelastic material, such as a nickel-titanium alloy, spring steel, or a polymeric material. Alternatively, stent 132 may be fabricated with a resilient knit or wickered weave pattern of elastic materials, such as stainless steel. If balloon-expandable, metal mesh, or bistable cell, stent 132 is preferably fabricated from elastic materials, such as stainless steel or titanium.

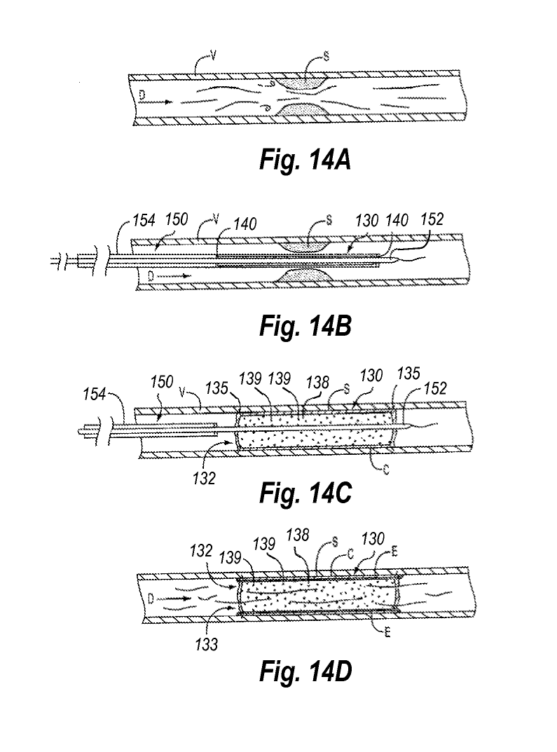

At least a portion of stent 132 preferably is radiopaque to facilitate proper positioning of apparatus 130 within a vessel. Alternatively, apparatus 130, or a delivery system for apparatus 130 (see FIG. 14), may comprise a radiopaque feature, for example, optional radiopaque marker bands 140, to facilitate positioning. Marker bands 140 comprise a radiopaque material, such as gold or platinum.

Apparatus 130 also may comprise coatings or attached active groups C configured for localized delivery of radiation, gene therapy, medicaments, thrombin inhibitors, or other therapeutic agents. Coatings or active groups C may, for example, be absorbed or adsorbed onto the surface, may be attached physically, chemically, biologically, electrostatically, covalently, or hydrophobically, or may be bonded to the surface through VanderWaal's forces, or combinations thereof, using a variety of techniques that are well-known in the art.

In FIG. 13A, apparatus 130 is shown in a collapsed delivery configuration, while, in FIG. 13B, apparatus 130 is in an expanded deployed configuration. If stent 132 is self-expanding, apparatus 130 may be collapsed to the delivery configuration over a guide wire or elongated member, and then covered with a sheath to maintain the apparatus in the delivery configuration. Using well-known percutaneous techniques, apparatus 130 is advanced through a patient's vasculature to a treatment site, where the sheath is withdrawn; stent 132 dynamically self-expands to the deployed configuration of FIG. 13B (see FIG. 14). If stent 132 is balloon expandable, apparatus 130 may be mounted in the delivery configuration on a balloon catheter, for delivery to the treatment site. Upon delivery using well-known techniques, the balloon catheter is inflated with sufficient pressure to facilitate irreversible expansion of the apparatus to the deployed configuration (not shown).

With reference to FIGS. 14A-14D, a method of using the apparatus of FIG. 13 within a patient's vasculature is described in greater detail. In FIG. 14, stent 132 of apparatus 130 is illustratively self-expanding. However, it should be understood that stent 132 alternatively may be, for example, balloon-expandable, bistable cell, or metal mesh, in accordance with the present invention.

In FIG. 14A, vessel V is partially occluded with stenosis S that disrupts blood flow in direction D. Using well-known techniques, apparatus 130, disposed in the collapsed delivery configuration over elongated member 152 and constrained in that configuration by sheath 154 of delivery system 150, is advanced to the point of stenosis, as seen in FIG. 14B. Radiopacity of stent 132, viewed under a fluoroscope, may facilitate proper positioning of apparatus 130 within the vessel. Alternatively, radiopaque marker bands 140, illustratively disposed on sheath 154, may facilitate positioning.

In FIG. 14C, sheath 154 is retracted proximally with respect to elongated member 152, thereby allowing apparatus 130 to dynamically self-expand to the deployed configuration. Apparatus 130 compresses and traps stenosis S against the wall of vessel V. Optional barb or hook 135 of stent 132 facilitates anchoring of stent 132 to vessel V. The controlled size of pores 139 along the length of apparatus 130 ensures that dangerous emboli, broken away from stenosis S during compression, do not escape from the vessel wall and enter the bloodstream. Apparatus 130 protects against embolization at the time of implantation, and further protects against delayed stroke caused by late embolization.

As seen in FIG. 14D, delivery system 150 is removed from the vessel. Pores 139 allow endothelial cells to rapidly migrate through apertures 136 of stent 132 and into the interior of apparatus 130 to form endothelial layer E over the entirety of apparatus 130. Layer E forms, for example, over a period of days to weeks. Unlike the endothelial layer covering stent graft 120 in FIG. 12, endothelial layer E of apparatus 130 is expected to form rapidly, to be complete, thin, and substantially regular. Layer E acts as a protective layer that reduces adverse interaction between apparatus 130 and the patient, thereby lessening the risk of thrombus formation and restenosis. Thus, in addition to maintaining patency of vessel V, apparatus 130 provides embolic protection coupled with reduced likelihood of restenosis and thrombus formation. Furthermore, optional coating or attached active groups C of material 138 may deliver radiation, gene therapy, medicaments, thrombin inhibitors, or other therapeutic substances to the vessel wall, or directly into the blood stream.

Apparatus 130 compresses and seals stenosis S against the wall of vessel V, thereby preventing embolic material from the stenosis from traveling downstream. Alternatively, via angioplasty or other suitable means, stenosis S may be compressed against the vessel wall prior to insertion of apparatus 130, in which case apparatus 130 still protects against delayed stroke caused by late embolization. In addition to the application of FIG. 14, apparatus 130 may be used for a variety of other applications, including, but not limited to, bridging defective points within a vessel, such as aneurysms, ruptures, dissections, punctures, etc.

While the rapid endothelialization of apparatus 130, discussed with respect to FIG. 14D, minimizes risk of restenosis and thrombus formation, restenosis may still occur in a limited number of patients. Additionally, vessel V may become lax and expand to a larger diameter. Under these and other circumstances, it may be necessary to recross lumen 133 of apparatus 130 with interventional instruments. These instruments may, for example, adjust apparatus 130, restore patency to vessel V in an area of restenosis, treat vascular complications distal to apparatus 130, or facilitate any of a variety of other minimally invasive procedures.

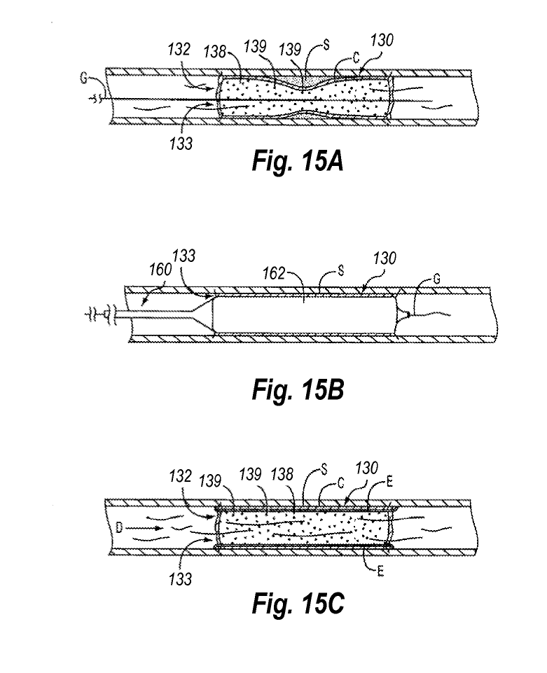

Referring now to FIGS. 15A-15C, capacity for recrossing with apparatus 130 is described. As in FIG. 14, stent 132 of apparatus 130 is illustratively self-expandable. In FIG. 15A, stent 132 has been implanted in vessel V using the techniques described hereinabove with respect to FIGS. 14A-14C. However, in contrast to FIG. 14C, stent 132 comprises insufficient radial strength to fully compress and seal stenosis S against the wall of vessel V. Guide wire G is therefore advanced through lumen 133 to provide a guide for advancement of a balloon catheter to fully compress stenosis S. The smooth interior surface provided by biocompatible material 138 of apparatus 130 ensures that guide wire G may recross lumen 133 without becoming entangled in the stent, as was described hereinabove with respect to FIG. 11B.

In FIG. 15B, once guide wire G has recrossed lumen 133, balloon catheter 160 is advanced over guide wire G to the point of stenosis S. Balloon 162 of catheter 160 is inflated with sufficient pressure to compress stenosis S against the walls of vessel V and fully deploy apparatus 130. As seen in FIG. 15C, balloon 162 is then deflated, and catheter 160 is removed from vessel V, thereby restoring patency to the vessel. Endothelial layer E then rapidly forms via endothelial cells that migrate through apertures 136 of stent 132 and pores 139 of material 138 into the interior of apparatus 130.

As will be apparent to those of skill in the art, recrossing of apparatus 130 may be indicated in a variety of applications, in addition to those of FIG. 15. For example, apparatus 130 may be recrossed in order to compress restenosis that has formed within the vessel, as illustrated with bare stent 114 in FIG. 11B. Additionally, apparatus 130 may be recrossed in order to resize the apparatus so that it conforms to, or accommodates changes in, vessel geometry.

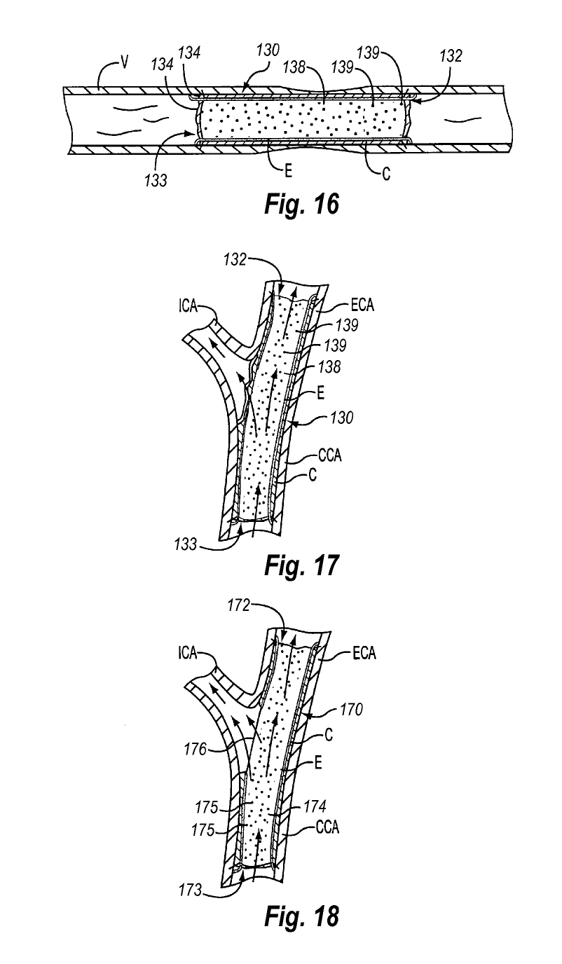

With reference now to FIG. 16, apparatus 130 has been implanted into vessel V that is undergoing temporary deformation, for example, due to contact with neighboring muscles, due to joint motion, or due to external pressure applied to the vessel. The wall of vessel V impinges on apparatus 130. In contrast to bare stent 114 of FIG. 11C, apparatus 130 distributes the load applied by vessel V across adjoining cells of structure 134 of stent 132, and across the section of biocompatible material 138 attached to the adjoining cells. Thus, the constricted portion of vessel V neither collapses within lumen 133 of apparatus 130 nor is punctured by apparatus 130. Additionally, since the load is distributed, stent 132 of apparatus 130 does not kink, and lumen 133 remains patent. Similarly, apparatus 130 is expected to continue to function safely and properly if implanted in tortuous anatomy.

Referring to FIG. 17, apparatus 130 is shown in use in a branched or bifurcated vessel. Using well-known techniques, apparatus 130 has been expanded to the deployed configuration within common carotid artery CCA and external carotid artery ECA. Internal carotid artery ICA branches off from the common carotid. Uninterrupted and unimpeded blood flow through the side branch presented by internal carotid artery ICA must be maintained when stenting in the common carotid artery CCA and external carotid artery ECA. Since pores 139 of biocompatible material 138 render apparatus 130 permeable to blood flow, continued blood flow into internal carotid artery ICA is expected to continue. Optionally, the diameter, density, shape and/or packing arrangement of pores 139 may be selectively altered in the region of the vessel branching to ensure that adequate blood continues into the side branch.

Bare stents implanted at a vessel bifurcation may disrupt flow and create areas of stagnation susceptible to thrombus formation. Moreover, bare stents may provide inadequate embolic protection in some applications. The small diameter of pores 139, as compared to the diameter of apertures 136 of stent 132, provides a grating that is expected to reduce turbulence and allow thrombus-free blood flow into the side branch.

Referring now to FIG. 18, an alternative embodiment of the present invention is shown in use at a vessel bifurcation. Apparatus 170 is similar to apparatus 130 of FIGS. 13-17, except that apparatus 170 comprises radial opening 176 that is expected to allow unimpeded blood flow to a vessel side branch at the point of stenting. Apparatus 170 comprises balloon-expandable or self-expanding stent 172 having lumen 173. Preferably, at least a portion of stent 172 is radiopaque. Biocompatible material 174 having pores 175 is attached to stent 172. Radial opening 176 extends through stent 172 and material. 174, thereby providing a side path for blood flow out of lumen 173.

Pores 175 of material 174 are sized such that apparatus 170 is impermeable to stenotic emboli larger than a predetermined size, but is permeable to rapid ingrowth of endothelial cells. Pores 175 preferably have a minimum width of approximately 30 μm and a maximum width of approximately 100 μm, and even more preferably have an average width of about 80 μm. Also, apparatus 170 may optionally comprise coating or attached active groups C, as discussed hereinabove with respect to apparatus 130.

In FIG. 18, apparatus 170 has been expanded to a deployed configuration within common carotid artery CCA and external carotid artery ECA. Prior to expansion of apparatus 170, radial opening 176 was aligned with internal carotid artery ICA to ensure uninterrupted and unimpeded blood flow through the side branch. In addition to maintenance of flow, apparatus 170 provides enhanced embolic protection, facilitates rapid endothelialization, and reduces the risk of restenosis and thrombus formation.

Prior to expansion of apparatus 170, radiopacity of stent 172, or other radiopaque features associated with apparatus 170, may facilitate the alignment of opening 176 with the side branch. Alternatively, Intravascular Ultrasound (“IVUS”) techniques may facilitate imaging and alignment. In this case, the delivery catheter for apparatus 170 also may comprise IVUS capabilities, or an IVUS catheter may be advanced into the vessel prior to expansion of apparatus 170 (not shown). Magnetic Resonance Imaging (“MRI”) or Optical Coherence Tomography (“OCT”), as well as other imaging modalities that will be apparent to those of skill in the art, alternatively may be used.

Additional embodiments of the present invention may be provided with a plurality of radial openings configured for use in vessels exhibiting a plurality of branchings. The present invention is expected to be particularly indicated for use in the carotid and femoral arteries, although embodiments also may find utility in a variety of other vessels, including the coronary and aortic arteries, and in non-vascular lumens, for example, in the biliary ducts, the respiratory system, or the urinary tract.

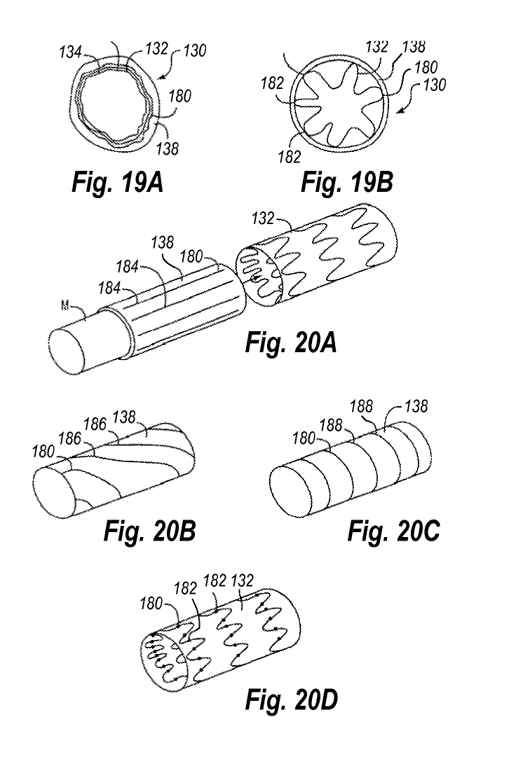

With reference now to FIGS. 19 and 20, exemplary techniques for manufacturing apparatus 130 of the present invention are provided. Other techniques within the scope of the present invention will be apparent to those of skill in the art.

Biocompatible material 138 preferably comprises a modified thermoplastic polyurethane, and even more preferably a siloxane modified thermoplastic polyurethane. The material preferably has a hardness in the range of about 70 A to 60 D, and even more preferably of about 55 D. Other materials and hardnesses will be apparent to those of skill in the art. Material 138 preferably is formed by a spinning process (not shown), for example, as described in U.S. Pat. No. 4,475,972 to Wong, which is incorporated herein by reference. Material 138 is heated to form a viscous liquid solution that is placed in a syringe. The material is advanced by a piston or plunger through a fine nozzle, where the material flows out onto a rotating mandrel as fine fibers. The fine fibers form a fibrous mat or covering of biocompatible covering material 138 on the rotating mandrel. As material 138 cools, the fibers solidify, and adjacent, contacting fibers are sintered to one another. Controlling the number of layers of fiber that are applied to the rotating mandrel provides control over the porosity of material 138.

If material 138 is to be sintered to stent 132, this may be achieved by disposing the stent over the mandrel prior to laying down material 138 (not shown). Material 138 also may be attached to either the internal or external surface of stent 132. FIGS. 19 and 20 provide various attachment schemes for attaching material 138 to a surface of the stent.

In FIG. 19A, stent 132 is attached with adhesive 180 to material 138 along all or most of structure 134 of stent 132. Adhesive 180 may comprise, for example, a material similar to biocompatible material 138, but with a different melting point. For example, adhesive 180 may comprise a modified thermoplastic polyurethane with a hardness of about 80 A. Stent 132 is dipped in the adhesive and dried. Then, stent 132 and material 138 are coaxially disposed about one another, and the composite apparatus is heated to a temperature above the melting point of adhesive 180, but below the melting point of biocompatible material 138. The composite apparatus is then cooled, which fuses material 138 to stent 132, thereby forming apparatus 130.

A drawback of the attachment scheme of FIG. 19A is that the quantity of adhesive used in forming apparatus 130 may add a significant amount of material to the apparatus, which may increase its delivery profile and/or its rigidity. Additionally, a risk may exist of adhesive particles coming loose during collapse or expansion of apparatus 130. If released within a patient's vasculature, these particles may act as emboli.

FIG. 19B provides an alternative attachment scheme. Material 138 is attached with adhesive 180 to stent 132 at discrete points 182, or is attached along defined planes, such as circumferential bands, longitudinal seams, or helical seams (see FIG. 20). Such attachment reduces the amount of adhesive material required, which, in turn, may reduce rigidity, delivery profile, and a risk of embolization of adhesive particles.

Referring to FIG. 20, various techniques for attaching a stent covering to a stent, in a manner that provides the attachment scheme of FIG. 19B, are provided. In FIGS. 20A-20C, biocompatible material 138 is configured for disposal along an interior surface of stent 132. Obviously, the material may alternatively be prepared for disposal about an exterior surface of the stent.

In FIG. 20A, biocompatible material 138 has been formed on mandrel M. Material 138 then is coated with longitudinal seams 184 of adhesive 180, and stent 132 is loaded over the material. Adhesive 180 bonds stent 132 to material 138 along seams 184. In FIG. 20B, material 138 is provided with helical seams 186 of adhesive 180, while in FIG. 20C, material 138 is provided with circumferential bands 188 of adhesive 180. In FIG. 20D, stent 132 is provided with adhesive 180 at discrete points 182. Points 182 may be on either the internal or external surface of stent 132, and biocompatible material 138 then is loaded onto to either the internal or external surface respectively. Additional adhesive configurations will be apparent to those of skill in the art.

The present additionally generally relates to medical implants, such as stents, having a porous membrane and the methods of making such membranes and medical implants. In accordance with the present invention, polymer membranes are provided that have well-defined pores based on a controlled deposition of fibers onto a substrate. In this manner a permeable membrane having a predetermined pore size and distribution may be obtained.

Acute as well as late embolization are a significant threat during and after intravascular interventions such as stenting in saphenous vein grafts (SVG) and carotid arteries, where released particles can lead to major cardiac attacks or strokes, respectively. Covered stents for treatment of atherosclerotic lesions constructed according to the present invention comprise a porous membrane bonded to an exterior surface, and interior surface, or both, of a stent. Advantageously, the covered stent of the present invention may serve both to reduce embolization during an interventional procedure and prevent late embolization by tethering emboli and particles to the vessel wall.

The inventive membrane may be engineered to provide any of a number of design properties, including: single and multi-component material composition; loading of one or more physiological (bioactive) substances into the polymer matrix; predetermined isotropic or an-isotropic mechanical properties; and predetermined pore geometry.

In accordance with the principles of the present invention, polymeric material is deposited onto a computer-controlled movable substrate. Controlling the relative location and motion of the material source with regard to the deposition location on the substrate and process parameters, such as material flow and viscosity of the deposited material, permits generation of a multitude of different patterns for the membrane.

The porous membrane of the present invention is sufficiently strong and flexible for use in medical devices, and preferably comprises steps of extruding a continuous fiber-forming biocompatible polymeric material through a reciprocating extrusion head onto a substrate to form an elongated fiber. The fiber is deposited on the substrate in a predetermined pattern in traces having a width of from 5 to 500 μm, adjacent traces being spaced apart from each other a distance of between 0 and 500 μm.

Preferably, the fibers have a predetermined viscous creep that allows adjacent traces to bond to one another at predetermined contact points upon deposition. The number of overlapping or crossing fibers generally should be less than 5, preferably less than 4, and most preferably 1 or 2. When cured, the biocompatible material provides a stable, porous membrane.

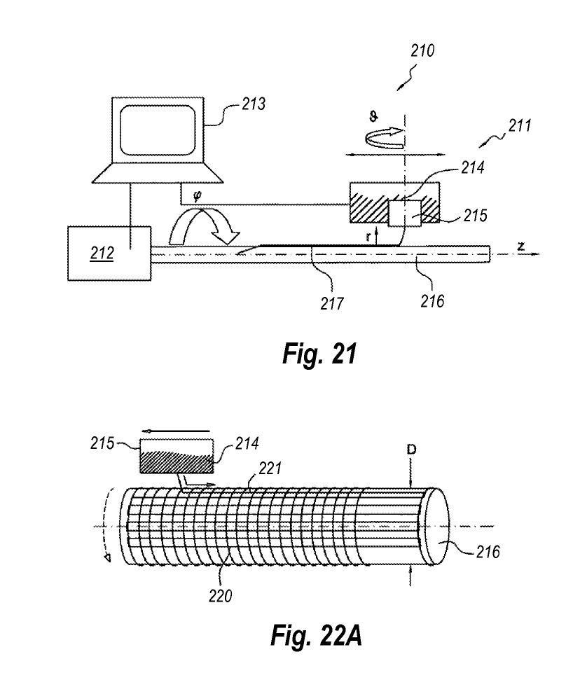

Referring to FIG. 21, apparatus 210 suitable for forming the porous membranes of the present invention comprises polymer extrusion machine 211 coupled to numerically controlled positioning system 212. Computer 213 controls the flow of extrudate 214 through extrusion head 215 as well as relative motion of extrusion head 215 and substrate 216 resulting from actuation of positioning system 212.

Apparatus 210 permits highly-localized deposition of the extrudate with four degrees of freedom onto a substrate to form a membrane. The degrees of freedom are: z—the longitudinal motion of substrate 216 relative to extrusion head 215; phi—the angular movement of substrate 216 relative to extrusion head 215; r—the distance between extrusion head 215 and substrate 216; and theta—the pivotal angle of extrusion head 215. The polymer strands 217 may be deposited onto the substrate under computer control to form any of the patterns described herein below.

In a preferred embodiment, the substrate comprises a rotating mandrel. Polymer is extruded through reciprocating extrusion head 215 representing the first degree of freedom z, and with a controlled distance between the extrusion head and substrate 216, representing the second degree of freedom r. Preferably, the distance between the extrusion head and substrate is between 0 to 50 mm, and more preferably between 0.5 and 20 mm. As the polymer is deposited onto the substrate, the substrate is rotated through a predetermined angle phi, corresponding to the third degree of freedom. In this manner, fibers 217 extruded from extrusion head 215 form a two-dimensional membrane on substrate 216. In addition, by pivoting the extrusion head along its vertical axis, fourth degree of freedom .theta. may be employed, thus making it possible to deposit more than one filament simultaneously while maintaining a set inter-fiber distance.

The four degrees of freedom discussed above may be independently controlled and if needed, synchronized, to attain a spatial resolution of material deposition having an order of magnitude of microns or higher. Optionally, the second degree of freedom r may be fixed if stable polymer deposition has been achieved. The fourth degree of freedom is not required when extruding only one filament.

Extrusion head 215 may have one or more outlets to deposit an extruded polymer fiber onto substrate 216 in traces having an inter-trace distance ranging between 0 to 1000 μm. The width of the individual trace (corresponding to the fiber width) may vary between 5 to 500 μm, and more preferably is in the range of 10 to 200 μm. Pore size is a function of trace width and inter-trace distance and may be selected by selection of these variables from between 0 (i.e., a tight covering) to 200 μm (i.e., to form a filter or tether to trap emboli against a vessel wall). Due to the precise control of fiber deposition, it is possible to create a membrane with desired porosity, strength and flexibility with a very small number of overlapping traces or crossing traces. The number of overlapping or crossing traces in the membrane of the present invention generally should be less than 5, preferably less than 4, and most preferably 1 to 2.

The biocompatible polymer is liquefied either by dissolving the biocompatible material in solvents or by thermally melting the biocompatible material, or both. The viscosity of the liquefied material determines the viscous creep properties and thus final pore size and inter-pore distance when the material is deposited on the substrate. Preferably, the viscous creep is controlled so that desired geometrical and physical properties are met upon deposition. By controlling the viscosity and amount of the deposited material on the substrate and consequently the viscous creep of the polymer before curing, the specified inter-pore distance, pore width and inter-fiber bonding may be achieved. Alternatively, the substrate may be heated to facilitate relaxation and/or curing of the trace width after deposition on the substrate.

Viscosity also may be controlled by adjustment of the distance r of extrusion head 215 relative to substrate 216, the concentration of the solvent in extrudate 214 and/or the heating temperature, ambient pressure, and extrusion parameters. With the viscous creep of the fibers being appropriately controlled, the traces deposited on the substrate will bond to one another at predetermined contact points upon deposition.

A specified pore size of the membrane may be achieved by, but is not limited to, lateral deposition distance between two adjacent material traces, extrusion parameters, and/or extrusion head outlet diameters and extrusion pressure. The latter two parameters also affect the fiber diameter, thus in combination with the fiber deposition pattern selected, permit selection and control of the mechanical properties of the membrane.

Suitable biocompatible materials include but are not limited to polyurethane and copolymers thereof, silicone polyurethane copolymer, polypropylene and copolymers thereof, polyamides, polyethylenes, PET, PEEK, ETFE, CTFE, PTFE and copolymers thereof. Preferred materials for forming membranes of the present invention are polyurethane and copolymers thereof. The polymers may in addition include any biologically active substance having desired release kinetics upon implantation into a patient's body.

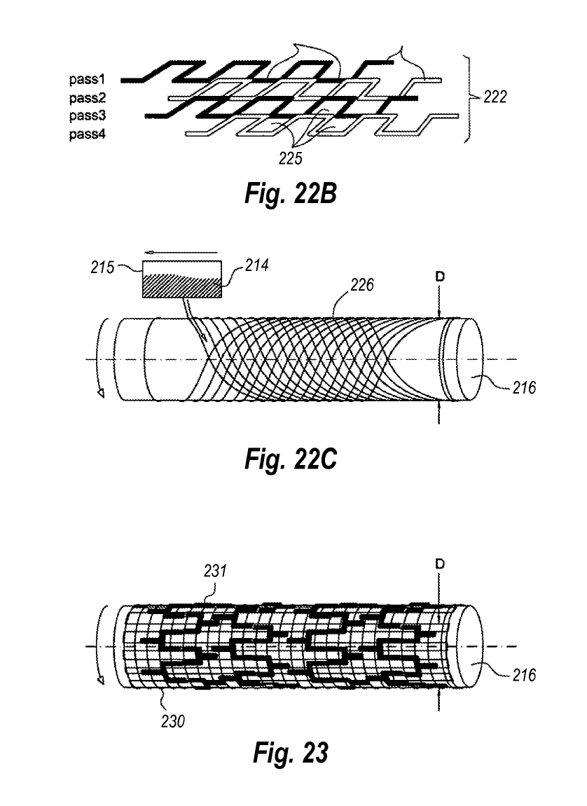

Referring now to FIGS. 22A to 22C, exemplary patterns formed by apparatus 210 during deposition of the fibers from extrusion head 215 of the present invention are described. In FIG. 22A, membrane 220 is formed on substrate 216 having diameter D by reciprocating extrusion head 215 longitudinally relative to the longitudinal axis of the substrate, followed by indexed angular movement of the substrate while the extrusion head is held stationary at the ends of the substrate. In this manner, traces 221 having a controlled width and inter-trace spacing may be deposited on the substrate.

Once the longitudinal fibers have been deposited on the substrate, the substrate is rotated 360.degree. while the extrusion head is indexed along the length of the substrate, thereby forming a regular pattern of square or rectangular pores having a predetermined size. Alternatively, if extrusion head 215 is provided with multiple outlets, multiple parallel fibers may be deposited in a single longitudinal pass.

FIG. 22B illustrates alternative membrane pattern 222, wherein the substrate is rotated through precise angular motions during longitudinal translation of the extrusion head. Instead of depositing a straight longitudinal strand, as in the pattern of FIG. 22A, the pattern of FIG. 22B includes a series of “jogs” 223 in each longitudinal filament 224. When adjacent filaments 224 are deposited on the substrate, the contacting portions of the traces bond to one another to define pores 225 having a predetermined size. In this manner, with each longitudinal pass of the extrusion head, a line of pores 225 of predetermined size in formed in a single layer membrane.

FIG. 22C shows another pattern 226 by which the membrane of the present invention may be built up. In this embodiment, positioning system 212 employs two degrees of freedom, z and phi, simultaneously, resulting in a “braid-like” structure. Preferably the extruded fibers retain a high unevaporated solvent content when deposited on substrate 216, so that the fibers fuse to form a unitary structure having a predetermined pore size.

More generally, apparatus 210 may be used to deposit one or more traces of a biocompatible material on substrate 216 while extrusion head 215 is reciprocated along the length of the substrate. An extrusion head having multiple outlets permit the deposition of multiple filaments on the substrate during a single translation of the extrusion head or rotation of the substrate. The multiple outlets may be arranged in any kind of required position on the extrusion head.” All translational and rotational motions of the components of apparatus 210 are individually or synchronously controlled by computer 213, thus permitting the membrane to be configured with any desired pattern.

As discussed above with respect to FIGS. 22A-22C, apparatus 210 permits fibers to be deposited with any of a number of possible alternative patterns. By depositing the fibers first in multiple passes longitudinal passes followed by indexed translation of the extrusion head and simultaneous rotation of the substrate, as in FIG. 22A, two trace layers may be generated that cross or overlap to form a membrane having a regular grid of pores. In this case, only one degree of freedom is used at any one time. Alternatively, addressing two degrees of freedom alternatingly, as in the pattern of FIG. 22B, a series of “jogs” may be introduced into the individual fibers. In this case, the traces do not cross but only contact each other, thereby creating a line of pores in a single layer membrane. Still further, by addressing two degrees of freedom simultaneously, a braided structure such as depicted in FIG. 22C may be obtained, in which a specified pore size and shape is attained by varying the distance between two parallel traces of material.

In accordance with one aspect of the present invention, extrusion is performed with chemically or thermally liquefied material, or both. The viscosity of the extrudate may be controlled by the concentration of the solvent, by enhancing evaporation of the solvent from the deposited material trace by means of heating the substrate, by varying the distance r between the extrusion head and the substrate, or by adjusting the extrusion temperature of the material so that a well-defined viscous creep of the material occurs after deposition onto the substrate.

Adjustment of the viscous creep allows fusion of the traces at contact points and thus formation of a two-dimensional membrane having desired mechanical strength characteristics. By appropriately setting these parameters accurate material deposition may be achieved with reduced lateral aberrations of the filaments compared to previously-known membrane manufacturing techniques.

As will of course be understood, the diameter of the substrate should be selected based upon the dimensions of the medical implant or stent to which the membrane is to be affixed. For example, the diameter may be selected based upon the expanded configuration of the medical implant or stent. The implant to be covered may be balloon-expandable or self-expandable. In a preferred embodiment, the implant is a self-expandable stent comprising a superelastic material such as a nickel-titanium alloy.

Referring to FIG. 23, stent 230 covered with membrane 231 of the present invention is described. Stent 230 may comprise any suitable design, such as a plastically deformable slotted tube or self-expanding superelastic structure. Porous membrane 231 may be deposited directly onto the medical implant, such as stent 230 which is employed as the substrate during the membrane deposition process.

Alternatively, the membrane may be deposited on a mandrel and after curing may be bonded in a separate step to the medical implant. In the latter case, thermal drying and/or evaporation of the solvent cures the biocompatible material while on the substrate. Once the membrane has cured sufficiently so that the mechanical properties of the membrane permit it to be removed from the substrate, the membrane may be bonded to a surface of the implant using a solvent, adhesive or thermal technique. In this case, the surface of the implant may be pre-processed to optimize bonding, for example by activation of the surface, coating of the surface with liquified polymer or other appropriate treatments of the surface.

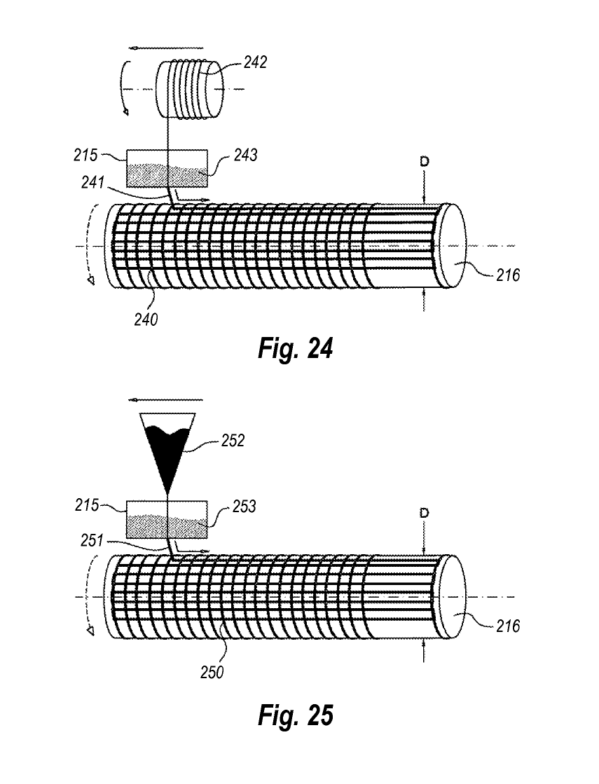

Referring now to FIG. 24, an alternative method of forming a porous membrane, suitable for use in a medical implant, is described. In this embodiment, membrane 240 comprises multi-component fiber 241 including core filament 242 coated with at least second biocompatible material 243 having the same or different chemical, physical, mechanical, biocompatible and or biologically active properties. Material 243 may incorporate one or more biologically active substances that elute into the patient's bloodstream after the medical implant is implanted.

Multi-component fiber 241 may be deposited onto the substrate to form a two-dimensional contiguous structure. The individual components of fiber 241 may be selected to provide different characteristics to the membrane, which may employ any of the pattern designs discussed herein above. For example, core filament 242 may provide mechanical stability, while material 243 may serve as an interface to the biological environment, enhance the adhesive properties for inter-trace bonding and/or enhance bonding of the membrane to the medical implant.

Suitable materials for the core filament include but are not limited to polyamides, polyethylenes, PET, PEEK, ETFE, CTFE, and PTFE and copolymers thereof, and metal wire or fiber glass. Suitable materials for ensheathing core filament 241 include but are not limited to polyurethane and copolymers thereof, silicone polyurethane copolymer, polypropylene and copolymers thereof, polyamides, polyethylenes, PET, PEEK, ETFE, CTFE, PTFE and copolymers thereof. These multi-component filaments allow performance of all the processes in membrane generation and all designs described above as well as achieve all the properties described in the other embodiments

Referring to FIG. 25, a further alternative method of forming the porous membrane of the present invention is described. In the method depicted in FIG. 25, membrane 250 comprises co-extruded fibers 251 formed of at least first biocompatible material 252 and second biocompatible material 253. Materials 252 and 253 may have the same or different chemical, physical, mechanical, biocompatible and or physiologically active properties. Fibers 251, while illustrated as being co-axially co-extruded, alternatively may be co-extruded co-linearly.

Suitable materials for first material 252 include but are not limited to polyamides, polyethylenes, PET, PEEK, ETFE, CTFE, PTFE and copolymers thereof. Suitable materials for second material 253 include but are not limited to polyurethane and copolymers thereof, silicone polyurethane copolymer, polypropylene and copolymers thereof, polyamides, polyethylenes, PET, PEEK, ETFE, CTFE, PTFE and copolymers thereof.

It should be understood that the present invention is not limited to membranes for use on stents. Rather, the membranes of the present invention may be affixed to any other medical device or implant that is brought into an intracorporal lumen for limited or extended implant durations. Such devices include vascular protection devices to filter emboli that are only transiently introduced into the body. Further applications for such porous membranes may be devices configured to be introduced into other body lumens or ducts, such as the trachea, esophagus, and biliary or urinary lumina.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What we claim is:

1. An endoprosthesis comprising:
a tubular expandable member, the tubular expandable member having a wall with an inner surface and an outer surface, the tubular expandable member additionally having proximal and distal ends and a lumen extending therebetween; and
a material having a plurality of pores, wherein the material is disposed about the tubular expandable member, at least some of the pores being permeable to endothelial cell ingrowth and impermeable to emboli larger than a predetermined size, at least some of the pores allowing continued blood flow therethrough.

2. The endoprosthesis of claim 1, wherein the material is formed by a weaving, knitting, or braiding process.

3. The endoprosthesis of claim 2, wherein the weaving, knitting, or braiding process determines pore sizes.

4. The endoprosthesis of claim 1, wherein the material is disposed about an outer surface of the tubular expandable member.

5. The endoprosthesis of claim 1, wherein disposed about the tubular expandable member comprises attached to at least a portion of the tubular member.

6. The endoprosthesis of claim 5, wherein attached comprises one or more of:
attaching with a bonding or sintering process;
attaching at least one discrete location along the tubular expandable member;
attaching along defined planes along the tubular expandable member; and
attaching along a majority of the tubular expandable member.

7. The endoprosthesis of claim 1, wherein the tubular expandable member has a radial opening for blood flow to a side branch vessel.

8. The endoprosthesis of claim 7, wherein the endoprosthesis has a lateral opening formed between the proximal and distal ends, and wherein the pores configured to allow blood flow therethrough are aligned with the radial opening.

9. The endoprosthesis of claim 8, wherein the pores aligned with the radial opening have a size different than the other pores.

10. The endoprosthesis of claim 1, wherein the pores which are permeable to endothelial cell ingrowth and impermeable to emboli larger than a predetermined size have a size between about 30 μm and 100 μm.

11. The endoprosthesis of claim 1, wherein the material comprises a biocompatible material selected from the group consisting of a biocompatible polymer, a modified thermoplastic polyurethane, polyethylene terephthalate, polyethylene tetraphthalate, expanded polytetrafluoroethylene, polypropylene, polyester, Nylon, polyethylene, polyurethane, a homologic material, an autologous or non-autologous vessel, a biodegradable material, polylactate, polyglycolic acid, or a combination thereof.

12. The endoprosthesis of claim 1, wherein the at least some of the plurality of pores have a larger size in the expanded configuration than in the collapsed configuration.

13. The endoprosthesis of claim 1, wherein the tubular expandable member is a stent.

14. The endoprosthesis of claim 1, wherein the endoprosthesis includes a coating, and wherein the coating
is configured to be absorbed or absorb on the surface of the material; and/or
comprises a therapeutic agent, wherein the therapeutic agent is chosen from the group consisting of attached active groups, radiation, gene vectors, medicaments, and thrombin inhibitors.

15. A method of making a porous membrane for use in medical implants comprising:
extruding a continuous fiber-forming biocompatible polymeric material through a reciprocating extrusion head to form an elongated fiber;
depositing the fiber onto a substrate in traces having a predetermined pattern and a trace width of 5 to 500 μm, adjacent traces being spaced apart a distance of between 0 and 500 μm, the fiber having a predetermined viscous creep characteristic that enables the adjacent traces to bond to each other at predetermined contact points; and
curing the biocompatible material on the substrate to provide a stable, porous membrane.

16. The method of claim 15 wherein depositing the fiber comprises one or more of:
depositing the fiber so that less than five adjacent traces of the fiber overlap or cross;
depositing the fiber so that adjacent traces of the fiber do not overlap or cross;
depositing the fiber so that adjacent traces of the fiber contact each other only at bond areas to define a row of pores; and
depositing the fiber with a high unevaporated solvent content so that adjacent traces of the fiber bond to each other

17. The method of claim 15 further comprising providing a substrate, wherein the substrate comprises a vascular implant.

18. The method of claim 17 further comprising providing a medical implant comprising a stent.

19. The method of claim 15 wherein extruding a continuous fiber-forming biocompatible polymeric material comprises co-extruding a polymeric sheath surrounding a solid core filament.

20. The method of claim 15 wherein extruding a continuous fiber-forming biocompatible polymeric material comprises co-extruding a first polymeric sheath surrounding a second polymeric core filament.

21. The method of claim 15 further comprising removing the porous membrane from the substrate and affixing the porous membrane to a surface of a medical implant.

22. Apparatus for making a porous membrane for use in medical implants, the apparatus comprising:
an extrusion head having an outlet for extruding a fiber comprising a biocompatible polymer;
a substrate;
a numerically-controlled positioning system configured to move the extrusion head relative to the substrate, the positioning system providing four degrees of freedom of movement of the extrusion head relative to the substrate; and
a computer coupled to control operation of the extrusion head and the positioning system.

23. The apparatus of claim 22 wherein:
a first degree of freedom comprises translational motion of the extrusion head relative to a longitudinal axis of the substrate;
a second degree of freedom comprises rotational motion of the extrusion head relative to a longitudinal axis of the substrate;
a third degree of freedom comprises varying a radial distance between the extrusion head and the substrate; and
a fourth degree of freedom comprises rotating the extrusion head relative to a vertical axis of the extrusion head.

24. The apparatus of claim 22 further comprising programming that controls the positioning system to rotate the substrate only when the extrusion head is stationary near a proximal or distal end of the substrate.

25. The apparatus of claim 22 further comprising programming that controls the positioning system to rotate the substrate in alternating directions when the extrusion head is disposed stationary at locations between a proximal end and a distal end of the substrate.

26. The apparatus of claim 22 further comprising programming the controls the positioning system to vary two or more degrees of freedom simultaneously.

27. The apparatus of claim 22 wherein the extrusion head is configured to:
extrude a fiber comprising a first polymer sheath co-extruded surrounding a core filament; and/or
co-linearly extrude multiple fibers.
